(19) United States
(12) Patent Application Publication
(10) Pub. No.: US 2023/0272293 A1
(43) Pub. Date: Aug. 31, 2023

(54) METHOD FOR PROCESSING PYROLYSIS OILS FROM PLASTICS AND/OR SOLID RECOVERED FUELS LOADED WITH IMPURITIES

(71) Applicant: IFP Energies nouvelles, Rueil-Malmaison Cedex (FR)

(72) Inventors: Wilfried WEISS, Rueil-Malmaison Cedex (FR); Alain QUIGNARD, Rueil-Malmaison Cedex (FR); Duc NGUYEN-HONG, Rueil-Malmaison Cedex (FR)

(73) Assignee: IFP Energies nouvelles, Rueil-Malmaison Cedex (FR)

(21) Appl. No.: 18/028,273

(22) PCT Filed: Sep. 9, 2021
(86) PCT No.: PCT/EP2021/074824

§ 371 Date: Mar. 24, 2023

(30) Foreign Application Priority Data
Sep. 25, 2020 (FR) 2009750

(51) Int. Cl. C10G 65/12; C07C 4/06

(52) U.S. Cl. CPC C10G 65/12; C07C 4/06; C10G 2300/301; C10G 2300/1003; C10G 2300/4006; C10G 2300/4012; C10G 2300/205; C10G 2300/202; C10G 2400/02

(57) ABSTRACT

The present invention relates to a process for treating an SRF and/or plastics pyrolysis oil, comprising:
a) optionally, selective hydrogenation of the feedstock;
b) hydroconversion in an ebullated bed, in an entrained bed and/or in a moving bed, to obtain a hydroconverted effluent;
c) separation of the hydroconverted effluent in the presence of an aqueous stream, to obtain a gaseous effluent, an aqueous liquid effluent and a liquid hydrocarbon effluent;
d) fractionation of the liquid hydrocarbon effluent to obtain at least one gas stream and a cut with a boiling point of less than or equal to 385° C. and a cut with a boiling point above 385° C.;
e) hydrotreatment of said cut comprising compounds with a boiling point of less than or equal to 385° C. to obtain a hydrotreated effluent;
f) separation to obtain at least a gaseous effluent and a hydrotreated liquid hydrocarbon effluent.

### TECHNICAL FIELD

The present invention relates to a process for treating a plastics pyrolysis oil and/or solid recovered fuels, loaded with impurities, so as to obtain a hydrocarbon effluent which can be upgraded by being at least partly incorporated directly into a naphtha or diesel pool or as feedstock for a steam cracking unit. More particularly, the present invention relates to a process for treating a feedstock obtained from the pyrolysis of plastic waste and/or SRF, so as to remove at least some of the impurities that said feedstock may contain in large amounts, and so as to hydrogenate the feedstock in order to be able to upgrade it.

### PRIOR ART

Plastics obtained from collection and sorting channels may undergo a step of pyrolysis so as to obtain, inter alia, pyrolysis oils. These plastics pyrolysis oils are generally burnt to generate electricity and/or used as fuel in industrial or urban heating boilers.

Solid recovered fuels (SRF), also called “refuse-derived fuel” or RDF, are solid non-hazardous waste prepared for energy recovery, whether they come from household and similar waste, waste from economic activities or construction and demolition waste. SRFs are generally a mixture of any combustible waste such as used tyres, food by-products (fats, animal meal, etc.), viscose and wood waste, light fractions from shredders (for example from used vehicles, electrical and electronic equipment (WEEE), household and commercial waste, residues from the recycling of various types of waste, including certain municipal waste, plastic waste, textiles, and wood among others. SRFs generally contain plastic waste. Nowadays, SRFs are mainly recovered as energy. They can be used directly as substitutes for fossil fuels in co-incineration facilities (coal and lignite power stations, cement works, lime kilns) or in household waste incineration units, or indirectly in pyrolysis units dedicated to energy recovery: SRF pyrolysis oils are thus generally burned to generate electricity, or even are used as fuel in industrial or urban heating boilers.

Another route for upgrading SRF and/or plastics pyrolysis oils is the use of these pyrolysis oils as feedstock for a steam cracking unit so as to (re)create olefins, said olefins being constituent monomers of certain polymers. However, plastic waste or SRFs are generally mixtures of several polymers, for example mixtures of polyethylene, polypropylene, polyethylene terephthalate, polyvinyl chloride and polystyrene. In addition, depending on the uses, plastics may contain, in addition to polymers, other compounds, such as plasticizers, pigments, dyes or polymerization catalyst residues, and also other very varied organic and mineral impurities from sorting centre separation operations, the selectivity of which operation may not be total.

Thus, the oils obtained from the pyrolysis of plastics or of SRFs comprise a lot of impurities, in particular diolefins, metals, silicon, or halogenated compounds, notably chlorine-based compounds, heteroelements such as sulfur, oxygen and nitrogen, and insoluble matter, in contents that are often high and incompatible with steam cracking units or units located downstream of the steam cracking units, notably polymerization processes and selective hydrogenation processes. These impurities may give rise to operability problems and notably problems of corrosion, coking or catalytic deactivation, or alternatively incompatibility problems in the applications of the target polymers. The presence of diolefins very often leads to problems of instability of the pyrolysis oil, characterized by the formation of gums. The gums and the insoluble matter that may be present in pyrolysis oil can give rise to problems of clogging in the processes.

Furthermore, during the steam cracking step, the yields of light olefins sought for petrochemistry, notably ethylene and propylene, depend greatly on the quality of the feedstocks sent for steam cracking. The BMCI (Bureau of Mines Correlation Index) is often used to characterize hydrocarbon cuts. This index, developed for hydrocarbon products derived from crude oils, is calculated from the measurement of the density and the average boiling point: it is equal to 0 for a linear paraffin and to 100 for benzene. Its value is therefore proportionately higher if the product analysed has an aromatic condensed structure, naphthenes having a BMCI intermediate between paraffins and aromatics. Overall, the yields of light olefins increase when the paraffin content increases and thus when the BMCI decreases. Conversely, the yields of undesired heavy compounds and/or of coke increase when the BMCI increases.

WO 2018/055555 proposes an overall process for recycling plastic waste, which is very general and relatively complex, ranging from the very step of pyrolysis of the plastic waste up to the steam cracking step. The process of patent application WO 2018/055555 comprises, inter alia, a step of hydrotreating the liquid phase obtained directly from the pyrolysis, preferably under quite stringent conditions notably in terms of temperature, for example at a temperature of between 260 and 300° C., a step of separation of the hydrotreatment effluent and then a step of hydrodealkylation of the heavy effluent separated out, preferably at a high temperature, for example between 260 and 400° C.

The unpublished patent application FR 20/01758 describes a process for treating a plastics pyrolysis oil, comprising:
a) selective hydrogenation of said feedstock in the presence of hydrogen and of a selective hydrogenation catalyst to obtain a hydrogenated effluent;
b) fixed-bed hydrotreatment of said hydrogenated effluent in the presence of hydrogen and of a hydrotreatment catalyst, to obtain a hydrotreatment effluent;
c) separation of the hydrotreatment effluent in the presence of an aqueous stream, at a temperature of between 50 and 370° C., to obtain a gaseous effluent, an aqueous liquid effluent and a liquid hydrocarbon effluent;
d) optionally, a step of fractionation of all or part of the hydrocarbon effluent obtained from step
c), to obtain a gas stream and at least two hydrocarbon streams which may be a naphtha cut and a heavier cut;
e) a recycling step comprising a phase of recovering a fraction of the hydrocarbon effluent obtained from the separation step c) or a fraction of and/or at least one of the hydrocarbon streams obtained from the fractionation step d), into the selective hydrogenation step a) and/or the hydrotreatment step b).

According to patent application FR 20/01758, the naphtha cut obtained from the fractionation step may be totally or partly sent either to a steam cracking unit or to a naphtha pool obtained from conventional petroleum feedstocks, or may be recycled into step e).

The heavier cut obtained from the fractionation step may be totally or partly sent either to a steam cracking unit or to a diesel or kerosene pool obtained from conventional petroleum feedstocks, or may be recycled into step e).

Unpublished patent applications FR 20/08108 and FR20/08106 are based on the process of FR20/01758 and describe a process for treating a plastics pyrolysis oil, incorporating one or two steps of hydrocracking in a fixed bed after the hydrotreatment step. These processes make it possible to minimize the yield of the heavy cut and to maximize the yield of the naphtha cut by transforming the heavy cut at least partly into naphtha cut by hydrocracking, which is the cut that is generally favoured for a steam cracking unit. Although the heavier cut can be sent to a steam cracking unit, few refiners favour this option. The reason for this is that the heavier cut has a high BMCI and contains, relative to the naphtha cut, more naphthenic, naphtheno-aromatic and aromatic compounds, thus leading to a higher C/H ratio. This high ratio is a cause of coking in the steam cracker, thus requiring steam cracking furnaces dedicated to this cut. Furthermore, the steam cracking of such a heavy cut produces a smaller amount of products of interest which are notably ethylene and propylene, but more pyrolysis gasoline.

Due to the content of impurities in pyrolysis oils, notably when they are heavily loaded with impurities, deactivation of the catalysts of the hydrotreatment unit which is operated in a fixed bed may be observed, which reduces the cycle time. Indeed, the main constraint of fixed-bed units is the fact that the unit has to be shut down to replace the catalysts. In addition, pyrolysis oils, notably those heavily loaded with impurities, can create clogging problems notably in preheating furnaces, feedstock/effluent exchangers or on the bed heads of catalytic reactors.

It would thus be advantageous to propose a process for treating pyrolysis oils having long-lasting catalytic cycles by allowing replacement of the catalysts without shutting down the unit, while at the same time producing a cut rich in alkanes which can be readily upgraded in a steam cracking unit.

Hydroconversion units operated with an ebullated bed, an entrained bed or even a moving bed are capable of processing this type of feedstock by virtue of a system for adding fresh catalyst and withdrawing spent catalyst without shutting down the unit. The addition of fresh catalyst and the withdrawal of spent catalyst are generally performed continuously, semi-continuously or periodically. These systems, which compensate for the deactivation of the catalysts due to impurities in the pyrolysates and solve the problems of clogging of the beds of catalysts of reactors operated with a fixed bed, allow the hydroconversion units to have a long cycle time without the need to shut down to replace the catalysts.

Furthermore, when such a hydroconversion unit is placed upstream of a hydrotreatment unit, the cycle time of the latter is increased by virtue of the hydrotreatment reactions performed partly beforehand in the hydroconversion unit.

Similarly, the hydrocracking reactions performed in the hydroconversion unit make it possible to transform at least some of the heavy compounds into lighter compounds, which makes it possible firstly to feed the hydrotreatment unit with a cut that is generally easier to process and secondly to obtain a cut having a lower BMCI which is thus particularly suitable for a steam cracking unit.

### SUMMARY OF THE INVENTION

The invention relates to a process for treating a feedstock comprising an SRF and/or plastics pyrolysis oil, comprising:
a) optionally, a selective hydrogenation step performed in a reaction section fed at least with said feedstock and a gas stream comprising hydrogen, in the presence of at least one selective hydrogenation catalyst, at a temperature of between 100 and 280° C., a partial pressure of hydrogen of between 1.0 and 20.0 MPa abs. and an hourly space velocity of between 0.3 and 10.0 h⁻¹, to obtain a hydrogenated effluent;
b) a hydroconversion step performed in a hydroconversion reaction section, using at least one ebullated-bed reactor, entrained-bed reactor or moving-bed reactor, comprising at least one hydroconversion catalyst, said hydroconversion reaction section being fed at least with said feedstock or with said hydrogenated effluent obtained on conclusion of step a) and a gas stream comprising hydrogen, said hydroconversion reaction section being operated at a temperature of between 250 and 450° C., a partial pressure of hydrogen of between 1.0 and 20.0 MPa abs. and an hourly space velocity of between 0.05 and 10.0 h⁻¹, to obtain a hydroconverted effluent;
c) a separation step, fed with the hydroconverted effluent obtained from step b) and an aqueous solution, said step being performed at a temperature of between 50 and 450° C., to obtain at least one gaseous effluent, an aqueous effluent and a hydrocarbon effluent;
d) a step of fractionating all or some of the hydrocarbon effluent obtained from step c), to obtain at least one gas stream, a hydrocarbon cut comprising compounds with a boiling point of less than or equal to 385° C. and a hydrocarbon cut comprising compounds with a boiling point above 385° C.,
e) a hydrotreatment step performed in a hydrotreatment reaction section, using at least one fixed-bed reactor containing n catalytic beds, n being an integer greater than or equal to 1, each comprising at least one hydrotreatment catalyst, said hydrotreatment reaction section being fed with at least some of said hydrocarbon cut comprising compounds with a boiling point of less than or equal to 385° C. obtained from step d) and a gas stream comprising hydrogen, said hydrotreatment reaction section being operated at a temperature of between 250 and 430° C., a partial pressure of hydrogen of between 1.0 and 20.0 MPa abs. and an hourly space velocity of between 0.1 and 10.0 h⁻¹, to obtain a hydrotreated effluent;
f) a separation step, fed with the hydrotreated effluent obtained from step e) to obtain at least a gaseous effluent and a hydrotreated liquid hydrocarbon effluent.

In the text hereinbelow, the term “pyrolysis oil” means an oil obtained from the pyrolysis of plastics and/or SRFs, unless otherwise indicated.

One advantage of the process according to the invention is that of purifying a pyrolysis oil of at least some of its impurities, which makes it possible to hydrogenate it and thus to be able to upgrade it in particular by incorporating it directly into a fuel pool and/or by making it compatible with a treatment in a steam cracking unit so as to be able in particular to obtain light olefins which may serve as monomers in the manufacture of polymers.

Another advantage of the invention is that of preventing risks of clogging and/or corrosion of the treatment unit in which the process of the invention is performed, the risks being exacerbated by the presence, often in large amounts, of diolefins, metals and halogenated compounds in the pyrolysis oil.

The process of the invention thus makes it possible to obtain a hydrocarbon effluent obtained from a pyrolysis oil which is at least partly freed of the impurities of the starting pyrolysis oil, thus limiting the problems of operability, such as the corrosion, coking or catalytic deactivation problems, to which these impurities may give rise, in particular in steam cracking units and/or in units located downstream of the steam cracking units, notably the polymerization and selective hydrogenation units. The removal of at least some of the impurities from the pyrolysis oils will also make it possible to increase the range of applications of the target polymers, the application incompatibilities being reduced.

The present invention participates in the recycling of plastics and/or SRFs, by proposing a process for treating an oil resulting from pyrolysis in order to purify it, hydroconvert it and hydrotreat it. Performing a hydroconversion step using a system for adding fresh catalyst and withdrawing used catalyst without shutting down the unit upstream of a fixed bed hydrotreatment step makes it possible in particular to treat pyrolysis oils that are heavily loaded with impurities.

Performing a hydroconversion step using a system for adding fresh catalyst and withdrawing used catalyst without shutting down the unit upstream of a fixed bed hydrotreatment step makes it possible not only to obtain long cycle times for hydroconversion but also makes it possible to lengthen the cycle time for the hydrotreatment step. In addition, the risk of clogging of the catalytic bed(s) of the hydrotreatment step is reduced.

Performing a hydroconversion step using a system for adding fresh catalyst and withdrawing used catalyst without shutting down the unit upstream of a fixed bed hydrotreatment step also makes it possible to convert at least some of the heavy compounds into lighter compounds, which makes it possible to obtain improved yields of the cut suitable for the steam cracking unit and, when this cut is sent for steam cracking, improved yields of light olefins, while at the same time reducing in particular the formation of coke in large quantities and/or the risks of corrosion encountered during subsequent step(s), for example during the step of steam cracking of the pyrolysis oils.

The oil fraction not converted by hydroconversion, corresponding to the hydrocarbon cut comprising compounds with a boiling point above 385° C. obtained from the fractionation step d), for its part, is preferably upgraded by recycling it into the hydroconversion step. Furthermore, the C2 to C4 compounds produced during the hydroconversion may also be sent for steam cracking, which makes it possible to improve the yields of light olefins (ethylene and propylene).

According to one variant, the process comprises said selective hydrogenation step a).

According to one variant, the hydrocarbon cut comprising compounds with a boiling point above 385° C. obtained from step d) is at least partly recycled into step b).

According to one variant, the process comprises a step a0) of pretreating the feedstock, said pretreatment step being performed upstream of the optional selective hydrogenation step a) or upstream of the hydroconversion step and comprises a filtration step and/or a step of washing with water and/or an adsorption step.

According to one variant, the hydrotreated liquid hydrocarbon effluent obtained from step f) is totally or partly sent into a steam cracking step h) performed in at least one pyrolysis furnace at a temperature of between 700 and 900° C. and at a pressure of between 0.05 and 0.3 MPa relative.

According to one variant, the process also comprises a recycling step g) in which a fraction of the hydrotreated liquid hydrocarbon effluent resulting from the separation step f) is sent into the optional selective hydrogenation step a) and/or the hydroconversion step b) and/or the hydrotreatment step e) and/or the hydrocracking step e′).

According to one variant, the separation step f) comprises a fractionation making it possible to obtain, in addition to a gas stream, a naphtha cut comprising compounds with a boiling point of less than or equal to 175° C., and a diesel cut comprising compounds with a boiling point above 175° C. and below 385° C.

According to one variant, the process also comprises a hydrocracking step e′) performed in a hydrocracking reaction section, using at least one fixed bed containing n catalytic beds, n being an integer greater than or equal to 1, each comprising at least one hydrocracking catalyst, said hydrocracking reaction section being fed at least with said hydrotreated effluent obtained from step e) and/or with the diesel cut comprising compounds with a boiling point above 175° C. and below 385° C. obtained from step f) and a gas stream comprising hydrogen, said hydrocracking reaction section being operated at a temperature of between 250 and 450° C., a partial pressure of hydrogen of between 1.5 and 20.0 MPa abs. and an hourly space velocity of between 0.1 and 10.0 h⁻¹, to obtain a hydrocracked effluent which is sent into the separation step f).

According to one variant, the separation step f) also comprises fractionation of the naphtha cut comprising compounds with a boiling point of less than or equal to 175° C. into a light naphtha cut comprising compounds having a boiling point below 80° C. and a heavy naphtha cut comprising compounds with a boiling point of between 80 and 175° C.

According to this variant, at least part of said heavy naphtha cut is sent to an aromatic complex including at least one naphtha reforming step and/or in which at least part of the light naphtha cut is sent into the steam cracking step h).

According to one variant, said selective hydrogenation catalyst of step a) comprises a support chosen from alumina, silica, silica-aluminas, magnesia, clays and mixtures thereof and a hydro-dehydrogenating function comprising either at least one group VIII element and at least one group VIB element, or at least one group VIII element.

According to one variant, when step b) is performed in an ebullated bed or in a moving bed, said hydroconversion catalyst of step b) comprises a supported catalyst comprising a group VIII metal chosen from the group formed by Ni, Pd, Pt, Co, Rh and/or Ru, optionally a group VIB metal chosen from the group of Mo and/or W, on an amorphous mineral support chosen from the group formed by alumina, silica, silica-aluminas, magnesia, clays and mixtures of at least two of these minerals, and when step b) is performed in an entrained bed, said hydroconversion catalyst of step b) comprises a dispersed catalyst containing at least one element chosen from the group formed by Mo, Fe, Ni, W, Co, V and Ru.

According to one variant, said hydrotreatment catalyst of step e) comprises a support chosen from the group consisting of alumina, silica, silica-aluminas, magnesia, clays and mixtures thereof and a hydro-dehydrogenating function comprising at least one group VIII element and/or at least one group VIB element.

According to one variant, said hydrocracking catalyst of step e′) comprises a support chosen from halogenated aluminas, combinations of boron and aluminium oxides, amorphous silica-aluminas and zeolites and a hydro-dehydrogenating function comprising at least one group VIB metal chosen from chromium, molybdenum and tungsten, alone or as a mixture, and/or at least one group VIII metal chosen from iron, cobalt, nickel, ruthenium, rhodium, palladium and platinum.

According to one variant, the feedstock has the following properties:
a content of aromatic compounds of between 0 and 90% by weight,
a content of halogenated compounds of between 2 and 5000 ppm by weight,
a content of metallic elements of between 10 and 10 000 ppm by weight,
including a content of iron element of between 0 and 100 ppm by weight,
a content of silicon element of between 0 and 1000 ppm by weight.

The invention also relates to the product which may be obtained via the treatment process according to the invention.

According to one variant, the product includes, relative to the total weight of the product:
a total content of metal elements of less than or equal to 5.0 ppm by weight,
including a content of iron element of less than or equal to 100 ppb by weight,
a content of silicon element of less than or equal to 1.0 ppm by weight,
a sulfur content of less than or equal to 500 ppm by weight,
a nitrogen content of less than or equal to 100 ppm by weight,
a content of chlorine element of less than or equal to 10 ppm by weight.

According to the present invention, the pressures are absolute pressures, also written as abs., and are given in MPa absolute (or MPa abs.), unless otherwise indicated.

According to the present invention, the expressions “included between . . . and . . . ” and “between . . . and . . . ” are equivalent and mean that the limit values of the interval are included in the described range of values. If such were not the case and if the limit values were not included in the described range, such a clarification will be given by the present invention.

For the purposes of the present invention, the various ranges of parameters for a given step, such as the pressure ranges and the temperature ranges, may be used alone or in combination. For example, for the purposes of the present invention, a range of preferred pressure values can be combined with a range of more preferred temperature values.

In the text hereinbelow, particular and/or preferred embodiments of the invention may be described. They may be performed separately or combined together without limitation of combination when this is technically feasible.

In the text hereinbelow, the groups of chemical elements are given according to the CAS classification (CRC Handbook of Chemistry and Physics, published by CRC Press, editor-in-chief D. R. Lide, 81^{st} edition, 2000-2001). For example, group VIII according to the CAS classification corresponds to the metals of columns 8, 9 and 10 according to the new IUPAC classification.

The content of metals is measured by X-ray fluorescence.

### DETAILED DESCRIPTION

The Feedstock

According to the invention, a “plastics pyrolysis oil or SRF pyrolysis oil” is an oil, advantageously in liquid form at room temperature, obtained from the pyrolysis of plastics, preferably of plastic waste notably originating from collection and sorting channels, or originating from the pyrolysis of SRFs. It comprises in particular a mixture of hydrocarbon compounds, notably paraffins, olefins, naphthenes and aromatics. At least 80% by weight of these hydrocarbon compounds preferably have a boiling point of less than 700° C., and preferably less than 550° C. In particular, depending on the origin of the pyrolysis oil, said oil may comprise up to 70% by weight of paraffins, up to 90% by weight of olefins and up to 90% by weight of aromatics, it being understood that the sum of the paraffins, of the olefins and of the aromatics is 100% by weight of the hydrocarbon compounds.

The density of the pyrolysis oil, measured at 15° C. according to the method ASTM D4052, is generally between 0.75 and 0.99 g/cm³, preferably between 0.75 and 0.95 g/cm³.

The pyrolysis oil may also comprise, and usually does comprise, impurities, such as metals, notably iron, silicon or halogenated compounds, notably chlorinated compounds. These impurities may be present in the pyrolysis oil in high contents, for example up to 500 ppm by weight or even 1000 ppm by weight or even 5000 ppm by weight of halogen elements provided by halogenated compounds, up to 2500 ppm by weight, or even 10 000 ppm by weight of metallic or semi-metallic elements. Alkali metals, alkaline-earth metals, transition metals, post-transition metals and metalloids may be likened to contaminants of metallic nature, referred to as metals or metallic or semi-metallic elements. The pyrolysis oil may comprise up to 200 ppm by weight or even 1000 ppm by weight of silicon, and up to 15 ppm by weight or even 100 ppm by weight of iron. The pyrolysis oil may also comprise other impurities such as heteroelements notably provided by sulfur compounds, oxygen compounds and/or nitrogen compounds, in contents generally less than 20 000 ppm by weight of heteroelements and preferably less than 10 000 ppm by weight of heteroelements.

The process according to the invention is particularly suitable for treating a pyrolysis oil loaded with impurities. This means a feedstock having the following properties:
an aromatic content of between 0 and 90% by weight, often between 20% and 90% by weight, and which may be between 50% and 90% by weight;
a halogen content of between 2 and 5000 ppm by weight, often between 200 and 5000 ppm by weight, and which may be between 500 and 5000 ppm by weight;
a content of metallic elements of between 10 and 10 000 ppm by weight, often between 2000 and 10 000 ppm by weight, and which may be between 2250 and 5000 ppm by weight;
including an iron element content of between 0 and 100 ppm by weight, often between 10 and 100 ppm by weight, and which may be between 15 and 100 ppm by weight;
a silicon element content of between 0 and 1000 ppm by weight, often between 100 and 1000 ppm by weight, and which may be between 200 and 1000 ppm by weight.

The process according to the invention is particularly suitable for treating a pyrolysis oil heavily loaded with impurities. This means a feedstock having the following properties:
a content of aromatic compounds of between 50% and 90% by weight;
a content of halogenated compounds of between 500 and 5000 ppm by weight;
a content of metallic elements of between 2250 and 10 000 ppm by weight;
including a content of iron element of between 15 and 100 ppm by weight;
a content of silicon element of between 200 and 1000 ppm by weight.

The feedstock of the process according to the invention comprises at least one SRF and/or plastics pyrolysis oil. Said feedstock may consist solely of plastics pyrolysis oil(s) or solely of SRF pyrolysis oil(s) or solely of a mixture of SRF and plastics pyrolysis oil(s). Preferably, said feedstock comprises at least 50% by weight, preferably between 50% and 100% by weight, and particularly preferably between 75% and 100% by weight of SRF and/or plastics pyrolysis oil.

The feedstock of the process according to the invention may also comprise a conventional petroleum-based feedstock and/or a feedstock obtained from the conversion of lignocellulose resources which is then co-treated with the SRF and/or plastics pyrolysis oil.

The SRF and/or plastics pyrolysis oil may be obtained from a thermal, catalytic pyrolysis treatment or else may be prepared by hydropyrolysis (pyrolysis in the presence of a catalyst and of hydrogen).

Pretreatment (Optional)

Said feedstock comprising a pyrolysis oil may advantageously be pretreated in an optional pretreatment step a0), prior to the optional selective hydrogenation step a) or the hydroconversion step b) when step a) is not present, to obtain a pretreated feedstock which feeds step a) or step b).

This optional pretreatment step a0) makes it possible to reduce the amount of contaminants, in particular the amount of silicon and of metals, which may be present in the feedstock comprising the pyrolysis oil. Thus, an optional step a0) of pretreatment of the feedstock comprising a pyrolysis oil may be performed in particular when said feedstock comprises more than 50 ppm by weight, notably more than 100 ppm by weight, more particularly more than 200 ppm by weight of metallic elements.

Said optional pretreatment step a0) may be performed via any method known to those skilled in the art for reducing the amount of contaminants. It may notably comprise a filtration step and/or a step of washing with water and/or an adsorption step.

According to one variant, said optional pretreatment step a0) is performed in an adsorption section operated in the presence of at least one adsorbent. Said optional pretreatment step a0) is performed at a temperature of between 0 and 150° C., preferably between 5 and 100° C., and at a pressure of between 0.15 and 10.0 MPa abs., preferably between 0.2 and 1.0 MPa abs. The adsorption section is advantageously operated in the presence of at least one adsorbent, preferably of alumina type, having a specific surface area of greater than or equal to 100 m²/g, preferably greater than or equal to 200 m²/g. The specific surface area of said adsorbent is advantageously less than or equal to 600 m²/g, in particular less than or equal to 400 m²/g. The specific surface area of the adsorbent is a surface area measured by the BET method, i.e. the specific surface area determined by nitrogen adsorption in accordance with the standard ASTM D 3663 established from the Brunauer-Emmett-Teller method described in the periodical The Journal of the American Chemical Society, 60, 309 (1938).

Advantageously, said adsorbent comprises less than 1% by weight of metallic elements, and is preferably free of metallic elements. The term “metallic elements of the adsorbent” should be understood as referring to the elements from groups 6 to 10 of the Periodic Table of the Elements (new IUPAC classification).

Said adsorption section of the optional step a0) comprises at least one adsorption column, preferably comprises at least two adsorption columns, preferentially between two and four adsorption columns, containing said adsorbent. When the adsorption section comprises two adsorption columns, one operating mode may be that referred to as “swing” operating according to the dedicated terminology, in which one of the columns is on-line, i.e. in service, while the other column is in reserve. When the adsorbent of the on-line column is spent, this column is isolated, while the column in reserve is placed on-line, i.e. in service. The spent adsorbent can then be regenerated in situ and/or replaced with fresh adsorbent so that the column containing it can once again be placed on-line once the other column has been isolated.

Another operating mode is to have at least two columns operating in series. When the adsorbent of the column placed at the head is spent, this first column is isolated and the spent adsorbent is either regenerated in situ or replaced with fresh adsorbent. The column is subsequently brought back on-line in the last position, and so on. This operation is known as permutable mode, or according to the term PRS for Permutable Reactor System, or also “lead and lag” according to the dedicated terminology. The combination of at least two adsorption columns makes it possible to overcome the possible and potentially rapid poisoning and/or clogging of the adsorbent due to the combined action of the metallic contaminants, of the diolefins, of the gums obtained from the diolefins and of the insoluble matter that may be present in the pyrolysis oil to be treated. The reason for this is that the presence of at least two adsorption columns facilitates the replacement and/or regeneration of the adsorbent, advantageously without stoppage of the pretreatment unit, or even of the process, thus making it possible to reduce the risks of clogging and thus to avoid stoppage of the unit due to clogging, to control the costs and to limit the consumption of adsorbent.

Said optional pretreatment step a0) may also optionally be fed with at least a fraction of a recycle stream, advantageously obtained from step g) of the process, as a mixture with or separately from the feedstock comprising a pyrolysis oil.

Said optional pretreatment step a0) thus makes it possible to obtain a pretreated feedstock which then feeds the selective hydrogenation step a) when it is present, or the hydroconversion step b).

Selective Hydrogenation Step a) (Optional)

According to the invention, the process may comprise a step a) of selective hydrogenation of the feedstock comprising a pyrolysis oil performed in the presence of hydrogen, under hydrogen pressure and temperature conditions making it possible to maintain said feedstock in the liquid phase and with an amount of soluble hydrogen which is just necessary for a selective hydrogenation of the diolefins present in the pyrolysis oil. Selective hydrogenation of the diolefins in liquid phase thus makes it possible to avoid or at least to limit the formation of “gums”, i.e. polymerization of the diolefins and thus the formation of oligomers and polymers, which can clog the reaction section of the hydrotreatment step e). Said selective hydrogenation step a) makes it possible to selectively obtain a hydrogenated effluent, i.e. an effluent with a reduced content of olefins, in particular of diolefins.

According to the invention, said selective hydrogenation step a) is performed in a reaction section fed at least with said feedstock comprising a pyrolysis oil, or with the pretreated feedstock obtained from the optional pretreatment step a0), and a gas stream comprising hydrogen (H₂). Optionally, the reaction section of said step a) may also be fed with at least a fraction of a recycle stream, advantageously obtained from the optional step g), either as a mixture with said feedstock, which has optionally been pretreated, or separately from the feedstock, which has optionally been pretreated, advantageously directly at the inlet of at least one of the reactors of the reaction section of step a). The introduction of at least a fraction of said recycle stream into the reaction section of the selective hydrogenation step a) advantageously makes it possible to dilute the impurities of the feedstock, which has optionally been pretreated, and to control the temperature notably in said reaction section.

Said reaction section involves selective hydrogenation, preferably in a fixed bed, in the presence of at least one selective hydrogenation catalyst, advantageously at a temperature of between 100 and 280° C., preferably between 120 and 260° C., preferably between 130 and 250° C., a partial pressure of hydrogen of between 1.0 and 20.0 MPa abs., preferably between 5.0 and 15.0 MPa abs. and at an hourly space velocity (HSV) of between 0.3 and 10.0 h⁻¹, preferably between 0.5 and 5.0 h⁻¹. The hourly space velocity (HSV) is defined here as the ratio of the hourly volume flow rate of the feedstock comprising the pyrolysis oil, which has optionally been pretreated, to the volume of catalyst(s). The amount of the gas stream comprising hydrogen (H₂) feeding said reaction section of step a) is advantageously such that the hydrogen coverage is between 1 and 200 Nm³ of hydrogen per m³ of feedstock (Nm³/m³), preferably between 1 and 50 Nm³ of hydrogen per m³ of feedstock (Nm³/m³), preferably between 5 and 20 Nm³ of hydrogen per m³ of feedstock (Nm³/m³). The hydrogen coverage is defined as the ratio of the volume flow rate of hydrogen taken under standard temperature and pressure conditions relative to the volume flow rate of “fresh” feedstock, i.e. of the feedstock to be treated, which has optionally been pretreated, without taking into account any recycled fraction, at 15° C. (in normal m³, written as Nm³, of H₂ per m³ of feedstock). The gas stream comprising hydrogen, which feeds the reaction section of step a), may consist of a supply of hydrogen and/or of recycled hydrogen obtained in particular from the separation step c).

The selective hydrogenation step a) is preferably performed in a fixed bed. It may also be performed in an ebullated bed or in a moving bed.

Advantageously, the reaction section of said step a) comprises between one and five reactors. According to a particular embodiment of the invention, the reaction section comprises between two and five reactors, which operate in permutable mode, referred to by the term PRS for permutable reactor system or by the term “lead and lag”. Combination of at least two reactors in PRS mode makes it possible to isolate one reactor, to discharge the spent catalyst, to recharge the reactor with fresh catalyst and to return said reactor into service without stopping the process. The PRS technology is described in particular in patent FR2681871.

Advantageously, reactor inserts, for example of filter plate type, may be used to prevent the clogging of the reactor(s). An example of a filter plate is described in patent FR3051375.

Advantageously, said selective hydrogenation catalyst comprises a support, preferably a mineral support, and a hydro-dehydrogenating function.

According to one variant, the hydro-dehydrogenating function in particular comprises at least one group VIII element, preferably chosen from nickel and cobalt, and at least one group VIB element, preferably chosen from molybdenum and tungsten. According to this variant, the total content of oxides of metallic elements from groups VIB and VIII is preferably between 1% and 40% by weight and preferentially between 5% and 30% by weight relative to the total weight of the catalyst. The weight ratio expressed as metal oxide between the group VIB metal(s) relative to the group VIII metal(s) is preferably between 1 and 20 and preferably between 2 and 10.

According to this variant, the reaction section of said step a) comprises, for example, a selective hydrogenation catalyst comprising between 0.5% and 12% by weight of nickel, preferably between 1% and 10% by weight of nickel (expressed as nickel oxide NiO relative to the weight of said catalyst), and between 1% and 30% by weight of molybdenum, preferably between 3% and 20% by weight of molybdenum (expressed as molybdenum oxide MoO₃ relative to the weight of said catalyst) on a support, preferably a mineral support, preferably on an alumina support.

According to another variant, the hydro-dehydrogenating function comprises, and preferably consists of, at least one group VIII element, preferably nickel. According to this variant, the content of nickel oxides is preferably between 1% and 50% by weight and preferably between 10% and 30% by weight relative to the weight of said catalyst. This type of catalyst is preferably used in its reduced form, on a support which is preferably mineral, preferably on an alumina support.

The support for said at least one selective hydrogenation catalyst is preferably chosen from alumina, silica, silica-aluminas, magnesia, clays and mixtures thereof. Said support may contain dopant compounds, notably oxides chosen from boron oxide, in particular boron trioxide, zirconia, ceria, titanium oxide, phosphorus pentoxide and a mixture of these oxides.

Preferably, said at least one selective hydrogenation catalyst comprises an alumina support, optionally doped with phosphorus and optionally boron. When phosphorus pentoxide P₂O₅ is present, its concentration is less than 10% by weight relative to the weight of the alumina and advantageously at least 0.001% by weight relative to the total weight of the alumina. When boron trioxide B₂O₅ is present, its concentration is less than 10% by weight relative to the weight of the alumina and advantageously at least 0.001% relative to the total weight of the alumina. The alumina used may be, for example, a γ(gamma) or η(eta) alumina.

Said selective hydrogenation catalyst is, for example, in the form of extrudates.

Very preferably, in order to hydrogenate the diolefins as selectively as possible, step a) may also use, in addition to the selective hydrogenation catalysts described above, at least one selective hydrogenation catalyst used in step a) comprising less than 1% by weight of nickel and at least 0.1% by weight of nickel, preferably 0.5% by weight of nickel, expressed as nickel oxide NiO relative to the weight of said catalyst, and less than 5% by weight of molybdenum and at least 0.1% by weight of molybdenum, preferably 0.5% by weight of molybdenum, expressed as molybdenum oxide MoO₃ relative to the weight of said catalyst, on an alumina support. This catalyst sparingly charged with metals is preferably placed upstream of the selective hydrogenation catalysts described above.

Optionally, the feedstock which comprises a pyrolysis oil, which has optionally been pretreated, and/or optionally mixed beforehand with at least one fraction of a recycle stream advantageously obtained from the optional step g), can be mixed with the gas stream comprising hydrogen prior to its introduction into the reaction section.

Said feedstock, which has optionally been pretreated, and/or optionally mixed with at least a fraction of the recycle stream, advantageously obtained from the optional step g), and/or optionally as a mixture with the gas stream, may also be heated before being introduced into the reaction section of step a), for example by heat exchange notably with the hydroconverted effluent from step b), to reach a temperature close to the temperature applied in the reaction section which it feeds.

The content of impurities, in particular of diolefins, of the hydrogenated effluent obtained on conclusion of step a) is reduced relative to that of the same impurities, in particular of diolefins, included in the feedstock for the process. The selective hydrogenation step a) generally makes it possible to convert at least 90% and preferably at least 99% of the diolefins contained in the initial feedstock. Step a) also makes it possible to remove, at least partly, other contaminants, for instance silicon. The hydrogenated effluent, obtained on conclusion of the selective hydrogenation step a), is sent, preferably directly, into the hydroconversion step b).

Hydroconversion Step b)

According to the invention, the treatment process comprises a hydroconversion step b) performed in a hydroconversion reaction section, involving at least one ebullated-bed reactor, entrained-bed reactor and/or moving-bed reactor, comprising at least one hydroconversion catalyst, said hydroconversion reaction section being fed at least with said feedstock or with said hydrogenated effluent obtained from step a), optionally mixed with at least a fraction of a recycle stream, advantageously obtained from the optional step g) and a gas stream comprising hydrogen, to obtain a hydroconverted effluent.

Advantageously, step b) involves hydroconversion reactions well known to those skilled in the art, and more particularly hydrotreating reactions such as the hydrogenation of olefins, aromatics, halogenated compounds, hydrodemetallization, hydrodesulfurization, hydrodeazotization, etc. and hydrocracking reactions (HCK) which lead to the opening of the naphthenic ring or the fractionation of paraffins into several fragments of lower molecular weight, thermal cracking and polycondensation reactions (formation of coke) although the latter are not desired.

Said hydroconversion reaction section of step b) may also be fed with at least a fraction of the recycle stream, advantageously obtained from the optional step g). Said fraction(s) of said recycle stream or the total amount of the recycle stream may be introduced into said hydroconversion reaction section as a mixture with the feedstock or the hydrogenated effluent obtained from step a) or separately. The introduction of at least a fraction of said recycle stream advantageously makes it possible to dilute the impurities still present in the hydrogenated effluent and to control the temperature, in particular to limit the increase in temperature, in the catalytic bed(s) of the hydroconversion reaction section which involves highly exothermic reactions.

Optionally, step b) may involve a heating section located upstream of the hydroconversion reaction section and in which the feedstock or the hydrogenated effluent obtained from step a) is heated to reach a temperature suitable for the hydroconversion, i.e. a temperature of between 250 and 450° C. Said optional heating section may thus comprise one or more exchangers, preferably allowing heat exchange between the feedstock or the hydrogenated effluent and the hydroconverted effluent, and/or a preheating furnace.

Advantageously, said hydroconversion reaction section is operated at a pressure equivalent to that used in the reaction section of the selective hydrogenation step a) when it is present, but at a higher temperature than that of the reaction section of the selective hydrogenation step a). Thus, said hydroconversion reaction section, this being true regardless of whether an ebullated bed, entrained bed and/or moving bed reaction section is used, is advantageously operated at a hydroconversion temperature of between 250 and 450° C., preferably between 350 and 420° C., at a partial pressure of hydrogen of between 1.0 and 20.0 MPa abs., more preferentially between 5.0 and 15.0 MPa abs., and at an hourly space velocity (HSV) of between 0.05 and 10.0 h⁻¹, preferably between 0.1 and 5.0 h⁻¹. According to the invention, the “hydroconversion temperature” corresponds to an average temperature in the hydroconversion reaction section of step b). The hydroconversion temperature is advantageously determined as a function of the catalytic systems, of the equipment and of the configuration thereof, by a person skilled in the art. For example, the ebullated bed hydroconversion temperature is determined by taking the arithmetic mean of the temperature measurements in the catalytic bed. The hourly space velocity (HSV) is defined here as the ratio of the hourly volume flow rate of the hydrogenated effluent obtained from step a) per volume of catalyst(s). The hydrogen coverage in step b) is advantageously between 50 and 1000 Nm³ of hydrogen per m³ of fresh feedstock which feeds step a), preferably between 60 and 500 Nm³ of hydrogen per m³ of fresh feedstock which feeds step a), preferably between 100 and 300 Nm³ of hydrogen per m³ of fresh feedstock which feeds step a). The hydrogen coverage is defined here as the ratio of the volume flow rate of hydrogen taken under standard temperature and pressure conditions relative to the volume flow rate of fresh feedstock which feeds step a), i.e. of feedstock comprising a pyrolysis oil, or with the feedstock which has optionally been pretreated, which feeds step a) (in normal m³, written as Nm³, of H₂ per m³ of fresh feedstock). The hydrogen may consist of a supply and/or of recycled hydrogen obtained in particular from the separation step c).

An important characteristic of the process according to the invention is the fact that the hydroconversion step is performed in a reaction section allowing the addition of fresh catalyst and the withdrawal of spent catalyst without shutting down the unit. Such systems are hydroconversion units operated in an ebullated bed, in an entrained bed and/or even in a moving bed. The addition of fresh catalyst and withdrawal of spent catalyst can thus be performed continuously, semi-continuously or periodically.

Ebullated-Bed Hydroconversion Step b)

Thus, according to a first variant, the hydroconversion step b) is performed in a hydroconversion reaction section involving at least one ebullated bed reactor. The functioning of the ebullated bed reactor, including the recycling of reactor liquids upwards through the stirred bed of catalyst, is generally well known. A mixture of feedstock and hydrogen is passed from the bottom upwards over a bed of catalytic particles at a flow rate such that the particles are subjected to a forced random motion whereas the liquid and gas pass through the bed from the bottom upwards. The movement of the catalytic bed is controlled by a flow of recycle liquid so that, in the steady state, the mass of the catalyst does not rise above a definable level in the reactor. Vapours and the liquid being hydrogenated pass through the upper level of the bed of catalytic particles to reach a zone substantially free of catalyst, and they are then discharged from the upper part of the reactor. A fraction of the reactor liquids is continuously recycled into the reactor. Ebullated bed technologies use supported catalysts, generally in the form of extrudates or beads whose diameter is generally of the order of 1 mm or less than 1 mm. The catalysts remain inside the reactors and are not discharged with the products. The catalytic activity can be kept constant by on-line replacement of the catalyst. It is thus not necessary to shut down the unit in order to change the spent catalyst, or to increase the reaction temperatures along the cycle in order to compensate for deactivation. Furthermore, working under constant operating conditions makes it possible to obtain constant product yields and qualities along the cycle. Also, because the catalyst is kept in agitation by a significant recycling of liquid, the pressure drop on the reactor remains low and constant, and the reaction exotherms are rapidly averaged over the catalytic bed.

The spent catalyst is partly replaced with fresh catalyst by withdrawal from the bottom of the reactor and introducing, either at the top of the reactor or at the bottom of the reactor, fresh or new catalyst at regular time intervals, that is to say by example in bursts or almost continuously.

Fresh catalyst can be introduced, for example, every day. The rate of replacement of the spent catalyst with fresh catalyst may be, for example, from about 0.01 kilogram to about 10 kilograms per cubic metre of feedstock. This withdrawal and this replacement are performed using devices which enable continuous functioning of this hydroconversion step. The unit usually includes an internal recirculation pump for maintaining the catalyst in an ebullated bed by continuous recycling of at least a portion of the liquid withdrawn at the top of the reactor and reinjected into the bottom of the reactor. It is also possible to send the spent catalyst withdrawn from the reactor to a regeneration zone, in which the carbon and sulfur which it contains are removed, and then to return this regenerated catalyst into the hydroconversion step. It is also possible to send the regenerated catalyst to a rejuvenation zone in which a treatment is performed aimed at improving the activity of the catalyst (presulfurization, additivation, etc.), then to return this rejuvenated catalyst into the hydroconversion step.

Catalysts used in an ebullated bed are widely marketed. These are granular catalysts whose size never reaches that of the catalysts used in an entrained bed. The catalyst is usually in the form of extrudates or beads. Typically, they contain at least one hydro-dehydrogenating element deposited on an amorphous support. Generally, the supported catalyst comprises a group VIII metal chosen from the group formed by Ni, Pd, Pt, Co, Rh and/or Ru, optionally a group VIB metal chosen from the group Mo and/or W, on an amorphous mineral support chosen from the group formed by alumina, silica, silica-aluminas, magnesia, clays and mixtures of at least two of these minerals. CoMo/alumina and NiMo/alumina catalysts are the most common.

The total content of oxides of metallic elements from groups VIB and VIII is preferably between 0.1% and 40% by weight and preferentially from 5% to 35% by weight relative to the total weight of the catalyst. The weight ratio expressed as metal oxide between the group VIB metal(s) relative to the group VIII metal(s) is preferably between 1.0 and 20 and preferably between 2.0 and 10. For example, the hydroconversion reaction section of step b) of the process comprises a hydroconversion catalyst comprising between 0.5% and 10% by weight of nickel, preferably between 1% and 8% by weight of nickel, expressed as nickel oxide NiO relative to the total weight of the hydroconversion catalyst, and between 1.0% and 30% by weight of molybdenum, preferably between 3.0% and 29% by weight of molybdenum, expressed as molybdenum oxide MoO₃ relative to the total weight of the hydroconversion catalyst, on a mineral support, preferably on an alumina support.

The support for said hydroconversion catalyst is advantageously chosen from alumina, silica, silica-aluminas, magnesia, clays and mixtures thereof. Said support may also contain dopant compounds, notably oxides chosen from boron oxide, in particular boron trioxide, zirconia, ceria, titanium oxide, phosphorus pentoxide and a mixture of these oxides. Preferably, said hydroconversion catalyst comprises an alumina support, preferably an alumina support doped with phosphorus and optionally boron. When phosphorus pentoxide P₂O₅ is present, its concentration is less than 10% by weight relative to the weight of the alumina and advantageously at least 0.001% by weight relative to the total weight of the alumina. When boron trioxide B₂O₅ is present, its concentration is less than 10% by weight relative to the weight of the alumina and advantageously at least 0.001% relative to the total weight of the alumina. The alumina used may be, for example, a γ(gamma) or η(eta) alumina.

Said hydroconversion catalyst is, for example, in the form of extrudates or beads.

Advantageously, said hydroconversion catalyst used in step b) of the process has a specific surface area of greater than or equal to 250 m²/g, preferably greater than or equal to 300 m²/g. The specific surface area of said hydroconversion catalyst is advantageously less than or equal to 800 m²/g, preferably less than or equal to 600 m²/g, in particular less than or equal to 400 m²/g. The specific surface area of the hydroconversion catalyst is measured by the BET method, i.e. the specific surface area determined by nitrogen adsorption in accordance with the standard ASTM D 3663 established from the Brunauer-Emmett-Teller method described in the periodical The Journal of the American Chemical Society, 60, 309 (1938). Such a specific surface area makes it possible to further improve the removal of the contaminants, in particular of the metals such as silicon.

Hydroconversion catalysts are distinguished from hydrotreatment catalysts notably by a porosity adapted to the treatment of impurities, notably metallic impurities, and in particular by the presence of macroporosity.

According to another aspect of the invention, the hydroconversion catalyst as described above also comprises one or more organic compounds containing oxygen and/or nitrogen and/or sulfur. Such a catalyst is often denoted by the term “additivated catalyst”. Generally, the organic compound is chosen from a compound including one or more chemical functions chosen from carboxylic, alcohol, thiol, thioether, sulfone, sulfoxide, ether, aldehyde, ketone, ester, carbonate, amine, nitrile, imide, oxime, urea and amide functions or else compounds including a furan ring or else sugars.

Entrained-Bed Hydroconversion Step b)

According to a second variant, the hydroconversion step b) is performed in a hydroconversion reaction section involving at least one entrained-bed reactor, also called a slurry reactor. The feedstock, hydrogen and the catalyst are injected from below and flow as an ascending stream.

The hydroconverted effluent and the unconsumed hydrogen and the catalyst are withdrawn from the top. The slurry hydroconversion technologies use a catalyst dispersed in the form of very small particles, the size of which is a few tens of microns or less (generally 0.001 to 100 μm). The catalysts, or the precursors thereof, are injected with the feedstock to be converted at the inlet of the reactors. The catalysts pass through the reactors with the feedstocks and the products undergoing conversion, and they are then entrained with the reaction products out of the reactors. They are found after separation in the heaviest fraction.

The slurry catalyst is a catalyst preferably containing at least one element chosen from the group formed by Mo, Fe, Ni, W, Co, V and Ru. These catalysts are generally monometallic or bimetallic (for example by combining a non-noble group VIIIB element (Co, Ni, Fe) and a group VIB element (Mo, W).

The catalysts used may be powders of heterogeneous solids (such as natural ores, iron sulfate, etc.), dispersed catalysts obtained from water-soluble precursors (“water soluble dispersed catalyst”) such as phosphomolybdic acid, ammonium molybdate, or a mixture of Mo or Ni oxide with aqueous ammonia.

Preferably, the catalysts used come from precursors that are soluble in an organic phase (“oil soluble dispersed catalyst”). The precursors are organometallic compounds such as naphthenates of Mo, Co, Fe, or Ni or such as multi-carbonyl compounds of these metals, for example 2-ethylhexanoates of Mo or Ni, acetylacetonates of Mo or Ni, salts of C7-C12 fatty acids of Mo or W, etc. They can be used in the presence of a surfactant to improve the dispersion of metals, when the catalyst is bimetallic.

The catalysts are in the form of dispersed particles, which may or may not be colloidal depending on the nature of the catalyst. Such precursors and catalysts that may be used in the process according to the invention are widely described in the literature.

The concentration of the catalyst, expressed as a metallic element, is generally between 1 and 10 000 ppm relative to the feedstock.

In general, the catalysts are prepared before being injected into the feedstock. The preparation process is adapted according to the state and nature of the precursor. In all cases, the precursor is sulfurized (ex-situ or in-situ) to form the catalyst dispersed in the feedstock.

For the preferred case of “oil-soluble” catalysts, in a typical process, the precursor is mixed with a carbon-based feedstock (which may be part of the feedstock to be treated, an external feedstock, a recycled fraction, etc.), the mixture is optionally at least partially dried, then or simultaneously sulfurized by adding a sulfur compound (H₂S preferred) and heated. The preparations of these catalysts are described in the prior art.

Additives can be added during the preparation of the catalyst or to the slurried catalyst before it is injected into the reactor. These additives are described in the literature.

The preferred solid additives are mineral oxides such as alumina, silica, mixed Al/Si oxides, supported spent catalysts (for example on alumina and/or silica) containing at least one group VIII element (such as Ni, Co) and/or at least one element of group VIB (such as Mo, W). Mention will be made, for example, of the catalysts described in patent application US 2008/177124. Carbon-based solids with a low hydrogen content (for example 4% hydrogen) such as coke, optionally pretreated, may also be used. Mixtures of such additives may also be used. Their particle sizes are preferably less than 1 mm. The content of any solid additive present at the inlet of the entrained bed hydroconversion reaction zone is between 0 and 10% by weight, preferentially between 1% and 3% by weight, and the content of the catalytic solutions is between 0 and 10% by weight, preferably between 0 and 1% by weight relative to the weight of the injected feedstock.

When the hydroconversion step b) is performed in an entrained bed reactor, a filtration step to recover the catalyst is necessary before sending the hydroconverted effluent into step c).

Moving-Bed Hydroconversion Step b)

According to a third variant, the hydroconversion step b) is performed in a hydroconversion reaction section involving at least one moving bed reactor.

The feedstock and the hydrogen can flow upward in moving bed reactors (countercurrent processes) or downward (cocurrent processes). The catalyst gradually flows by gravity from top to bottom and in plug flow inside the catalytic zone. It is withdrawn from below by any appropriate means, for example an elevator (called a “lift”). An in-line device ensures the semi-continuous renewal of the catalyst of the moving bed reactors: some of the spent catalyst is drawn off at the bottom of the reactor while fresh catalyst is introduced at the top of the reactor. The temperature is controlled therein by inter- or intra-reactor quenching.

Preferably, spherical catalysts with a diameter of between 0.5 and 6 mm and preferably between 1 and 3 mm are used rather than extruded catalysts, to obtain better flow. When the used catalyst is withdrawn from the bottom of the reactor, the entire catalytic bed moving in plug flow, moves downwards by a height corresponding to the volume of catalyst withdrawn. The degree of expansion of the catalytic bed operating as a moving bed is advantageously less than 15%, preferably less than 10%, preferably less than 5% and more preferably less than 2%. The degree of expansion is measured according to a method known to those skilled in the art.

The hydroconversion catalyst used in the moving bed of step b) of the process according to the invention is advantageously a catalyst comprising a support, preferably an amorphous support and very preferably alumina, and at least one group VIII metal chosen from nickel and cobalt, and preferably nickel, said group VIII element preferably being used in combination with at least one group VIB metal chosen from molybdenum and tungsten, and preferably the group VIB metal is molybdenum. Preferably, the hydroconversion catalyst comprises nickel as group VIII element and molybdenum as group VIB element. The nickel content is advantageously between 0.5% and 10% expressed by weight of nickel oxide (NiO) and preferably between 1% and 6% by weight, and the molybdenum content is advantageously between 1% and 30% expressed by weight of molybdenum trioxide (MoO₃), and preferably between 4% and 20% by weight, the percentages being expressed as weight percentage relative to the total weight of the catalyst. This catalyst is advantageously in the form of extrudates or beads. This catalyst may also advantageously contain phosphorus and preferably a content of phosphorus pentoxide P₂O₅ of less than 20% and preferably less than 10% by weight, the percentages being expressed as weight percentage relative to the total weight of the catalyst. The catalyst may also be a catalyst supplemented with an organic compound as described above.

According to yet another variant, the hydroconversion step b) may be performed in a hydroconversion reaction section involving a combination of at least one ebullated bed reactor, at least one entrained bed reactor and/or at least one moving bed reactor, in any order.

Preferably, step b) is performed in a hydroconversion reaction section involving at least one ebullated bed reactor.

Separation Step c)

According to the invention, the treatment process comprises a separation step c), advantageously performed in at least one washing/separation section, fed at least with the hydroconverted effluent obtained from step b) and an aqueous solution, to obtain at least one gaseous effluent, an aqueous effluent and a hydrocarbon effluent.

The gaseous effluent obtained on conclusion of step c) advantageously comprises hydrogen, preferably comprises at least 90% by volume, preferably at least 95% by volume, of hydrogen. Advantageously, said gaseous effluent may be at least partly recycled into the selective hydrogenation step a) and/or the hydroconversion step b) and/or the hydrotreatment step e) and/or the hydrocracking step e′), the recycling system possibly comprising a purification section.

The aqueous effluent obtained on conclusion of step c) advantageously comprises ammonium salts and/or hydrochloric acid. The aqueous effluent can be recycled into step c).

The separation step c) in particular makes it possible to remove the ammonium chloride salts which form by reaction between the chloride ions, released by hydrogenation of the chlorinated compounds notably in HCl form during step b) followed by dissolution in the water, and the ammonium ions, generated by hydrogenation of the nitrogenous compounds in the form of NH₃ notably during step b) and/or provided by injection of an amine followed by dissolution in the water, and thus to limit the risks of clogging, in particular in the transfer lines and/or in the sections of the process of the invention and/or the transfer lines to the steam cracker, due to the precipitation of the ammonium chloride salts. It also makes it possible to remove the hydrochloric acid formed by the reaction of the hydrogen ions and the chloride ions.

As a function of the content of chlorinated compounds in the initial feedstock to be treated, a stream containing an amine, for instance monoethanolamine, diethanolamine and/or monodiethanolamine, may be injected upstream of the selective hydrogenation step a), between the selective hydrogenation step a) and the hydroconversion step b) and/or between the hydroconversion step b) and the separation step c), preferably upstream of the selective hydrogenation step a) when it is present, so as to ensure a sufficient amount of ammonium ions to combine with the chloride ions formed during the hydroconversion step, thus making it possible to limit the formation of hydrochloric acid and thus to limit corrosion downstream of the separation section.

Advantageously, the separation step c) comprises an injection of an aqueous solution, preferably an injection of water, into the hydroconverted effluent obtained from step b), upstream of the washing/separation section, so as to at least partly dissolve the ammonium chloride salts and/or the hydrochloric acid and thus to improve the removal of the chlorinated impurities and to reduce the risks of clogging caused by an accumulation of the ammonium chloride salts.

The separation step c) is advantageously performed at a temperature of between 50 and 450° C., preferentially between 100 and 440° C., preferably between 200 and 420° C. It is important to perform said step in this temperature range (and therefore not to cool the hydroconverted effluent too much) at the risk of clogging in the lines due to the precipitation of the ammonium chloride salts. Advantageously, the separation step c) is performed at a pressure close to that used in steps a) and/or b), preferably between 1.0 and 20.0 MPa, so as to facilitate the recycling of hydrogen.

The washing/separation section of step c) may be at least partly performed in common or separate washing and separation equipment, this equipment being well known (separating vessels which may be operated at various pressures and temperatures, pumps, heat exchangers, washing columns, etc.).

In one embodiment of the invention, the separation step c) comprises the injection of an aqueous solution into the hydroconverted effluent obtained from step b), followed by the washing/separation section advantageously comprising a separation phase for obtaining at least one aqueous effluent charged with ammonium salts, a washed liquid hydrocarbon effluent and a partially washed gaseous effluent. The aqueous effluent charged with ammonium salts and the washed liquid hydrocarbon effluent may subsequently be separated in a decanting vessel so as to obtain said hydrocarbon effluent and said aqueous effluent. Said partially washed gaseous effluent can, in parallel, be introduced into a washing column where it circulates countercurrentwise to an aqueous stream, preferably of the same nature as the aqueous solution injected into the hydroconverted effluent, which makes it possible to remove, at least partly and preferably completely, the hydrochloric acid contained in the partially washed gaseous effluent and thus to obtain said gaseous effluent, preferably essentially comprising hydrogen, and an acidic aqueous stream. Said aqueous effluent obtained from the decanting vessel may optionally be mixed with said acidic aqueous stream, and be used, optionally as a mixture with said acidic aqueous stream, in a water recycling circuit to feed step c) of separation into said aqueous solution upstream of the washing/separation section and/or into said aqueous stream in the washing column. Said water recycling circuit may include a supply of water and/or of a basic solution and/or a purge for removing the dissolved salts.

In another embodiment of the invention, the separation step c) may advantageously comprise a “high-pressure” washing/separation section which operates at a pressure close to the pressure of the selective hydrogenation step a) and/or of the hydroconversion step b), preferably between 1.0 and 20.0 MPa, so as to facilitate the recycling of hydrogen. This “high pressure” section of step c) can be supplemented by a “low pressure” section, preferably a pressure generally between 0.5 and 10.0 MPa, so as to obtain a liquid hydrocarbon fraction free of a portion of the gases dissolved at high pressure and intended to be treated sent into the fractionation step d).

The gas fraction(s) obtained from the separation step c) may undergo additional purification(s) and separation(s) for the purpose of recovering at least one hydrogen-rich gas which may be recycled upstream of steps a) and/or b) and/or e) and/or e′) and light hydrocarbons, notably ethane, propane and butane, which may advantageously be sent separately or as a mixture into one or more furnaces of the steam cracking step h).

The hydrocarbon effluent obtained from the separation step c) is sent, partly or totally, preferably totally, into the fractionation step d).

Fractionation Step d)

The process according to the invention comprises a step of fractionating all or a portion, preferably all, of the hydrocarbon effluent obtained from step c), to obtain at least one gas stream, a hydrocarbon cut comprising compounds with a boiling point of less than or equal to 385° C., and a hydrocarbon cut comprising compounds with a boiling point above 385° C.

Step d) makes it possible in particular to remove the gases dissolved in the liquid hydrocarbon effluent, for instance ammonia, hydrogen sulfide and light hydrocarbons containing 1 to 4 carbon atoms.

The fractionation step d) is advantageously performed at a pressure of less than or equal to 1.0 MPa abs., preferably between 0.1 and 1.0 MPa abs.

According to one embodiment, step d) may be performed in a section advantageously comprising at least one stripping column equipped with a reflux circuit comprising a reflux vessel. Said stripping column is fed with the liquid hydrocarbon effluent obtained from step c) and with a steam stream. The liquid hydrocarbon effluent obtained from step c) may optionally be heated before entering the stripping column. Thus, the lightest compounds are entrained to the top of the column and into the reflux circuit comprising a reflux vessel in which a gas/liquid separation is performed. The gaseous phase which comprises the light hydrocarbons is withdrawn from the reflux vessel as a gas stream. The cut comprising compounds with a boiling point of less than or equal to 385° C. is advantageously withdrawn from the reflux vessel. The hydrocarbon cut comprising compounds with a boiling point of greater than 385° C. is advantageously withdrawn at the bottom of the stripping column.

According to other embodiments, the fractionation step d) may involve a stripping column followed by a distillation column or only a distillation column.

The cut comprising compounds with a boiling point of less than or equal to 385° C. (naphtha cut and diesel cut) is sent, totally or partly, into the hydrotreatment step e).

The cut comprising compounds with a boiling point above 385° C. (unconverted oils) is advantageously at least partly recycled into the hydroconversion step b). It can also be burned to produce heat and/or electricity.

A purge can be installed on the recycle of said cut comprising compounds with a boiling point above 385° C. obtained from step d). Depending on the operating conditions of the process, said purge may be between 0 and 50% by weight of said cut obtained from step d), and preferably between 5% and 20% by weight.

The gas fraction(s) obtained from the fractionation step d) may undergo additional purification(s) and separation(s) for the purpose of recovering at least light hydrocarbons, notably ethane, propane and butane, which may advantageously be sent separately or as a mixture into one or more furnaces of the steam cracking step h).

Hydrotreatment Step e)

According to the invention, the treatment process comprises a hydrotreatment step e) performed in a hydrotreatment reaction section, using at least one fixed-bed reactor containing n catalytic beds, n being an integer greater than or equal to 1, each comprising at least one hydrotreatment catalyst, said hydrotreatment reaction section being fed with at least a portion of said hydrocarbon cut comprising compounds with a boiling point of less than or equal to 385° C. obtained from step d) and a gas stream comprising hydrogen, to obtain a hydrotreated effluent.

Advantageously, step c) involves hydrotreatment reactions that are well known to those skilled in the art, and more particularly hydrotreatment reactions such as the hydrogenation of aromatics, hydrodesulfurization and hydrodeazotization. Furthermore, the hydrogenation of the olefins and of the remaining halogenated compounds and also the hydrodemetallation are continued.

Advantageously, said step e) is performed in a hydrotreatment reaction section comprising at least one, preferably between one and five, fixed-bed reactors containing n catalytic beds, n being an integer greater than or equal to one, preferably between one and ten, preferably between two and five, said bed(s) each comprising at least one and preferably not more than ten hydrotreatment catalysts. When a reactor comprises several catalytic beds, i.e. at least two, preferably between two and ten, preferably between two and five catalytic beds, said catalytic beds are arranged in series in said reactor.

Said hydrotreatment reaction section is fed with at least part of said hydrocarbon cut comprising compounds with a boiling point of less than or equal to 385° C. obtained from step d) and a gas stream comprising hydrogen, advantageously at the level of the first catalytic bed of the first reactor in operation.

Said hydrotreatment reaction section of step e) may also be fed with at least a fraction of the recycle stream, advantageously obtained from the optional step g). Said fraction(s) of said recycle stream or the total amount of the recycle stream may be introduced into said hydrotreatment reaction section as a mixture with said hydrocarbon cut comprising compounds with a boiling point of less than or equal to 385° C. obtained from step d) or separately. Said fraction(s) of said recycle stream or the total amount of the recycle stream may be introduced into said hydrotreatment reaction section into one or more catalytic beds of said hydrotreatment reaction section of step e). Introduction of at least a fraction of said recycle stream advantageously makes it possible to dilute the impurities still present in the hydrogenated effluent and to control the temperature, in particular to limit the temperature increase, in the catalytic bed(s) of the hydrotreatment reaction section which involves highly exothermic reactions.

Said hydrotreatment reaction section is advantageously operated at a hydrotreatment temperature of between 250 and 430° C., preferably between 300 and 400° C., at a partial pressure of hydrogen of between 1.0 and 20.0 MPa abs., preferably between 3.0 and 15.0 MPa abs., and at an hourly space velocity (HSV) of between 0.1 and 10.0 h⁻¹, preferably between 0.1 and 5.0 h⁻¹, preferentially between 0.2 and 2.0 h⁻¹, preferably between 0.2 and 1.0 h⁻¹. According to the invention, the “hydrotreatment temperature” corresponds to an average temperature in the hydrotreatment reaction section of step e). In particular, it corresponds to the weight-average bed temperature (WABT) according to the dedicated terminology, which is well known to those skilled in the art. The hydrotreatment temperature is advantageously determined as a function of the catalytic systems, of the equipment and of the configuration thereof that are used. For example, the hydrotreatment temperature (or WABT) is calculated in the following manner:

WABT=(Tᵢₙₗₑₜ+2×Tₒᵤₜₗₑₜ)/3

with Tᵢₙₗₑₜ: the temperature of the effluent with a boiling point of less than or equal to 385° C. at the inlet of the hydrotreatment reaction section, Tₒᵤₜₗₑₜ: the temperature of the effluent at the outlet of the hydrotreatment reaction section.

The hourly space velocity (HSV) is defined here as the ratio of the hourly volume flow rate of the hydrocarbon cut comprising compounds with a boiling point of less than or equal to 385° C. obtained from step d) per volume of catalyst(s). The hydrogen coverage in step e) is advantageously between 50 and 2000 Nm³ of hydrogen per m³ of feedstock which feeds step e), preferably between 100 and 1000 Nm³ of hydrogen per m³ of feedstock which feeds step e), preferably between 120 and 800 Nm³ of hydrogen per m³ of feedstock which feeds step e). The hydrogen coverage is defined here as the ratio of the volume flow rate of hydrogen taken under standard temperature and pressure conditions relative to the volume flow rate of feedstock which feeds step e) (in normal m³, written as Nm³, of H₂ per m³ of fresh feedstock). The hydrogen may consist of a supply and/or of recycled hydrogen obtained in particular from the separation step c).

Preferably, an additional gas stream comprising hydrogen is advantageously introduced into the inlet of each reactor, in particular operating in series, and/or into the inlet of each catalytic bed from the second catalytic bed of the hydrotreatment reaction section. These additional gas streams are also referred to as cooling streams. They make it possible to control the temperature in the hydrotreatment reactor in which the reactions involved are generally highly exothermic.

Advantageously, said hydrotreatment catalyst used in said step e) may be chosen from known hydrodemetallation, hydrotreatment or silicon scavenging catalysts notably used for the treatment of petroleum cuts, and combinations thereof. Known hydrodemetallation catalysts are, for example, those described in patents EP 0113297, EP 0113284, U.S. Pat. Nos. 5,221,656, 5,827,421, 7,119,045, 5,622,616 and 5,089,463. Known hydrotreatment catalysts are, for example, those described in patents EP 0113297, EP 0113284, U.S. Pat. Nos. 6,589,908, 4,818,743 or U.S. Pat. No. 6,332,976. Known silicon scavenging catalysts are, for example, those described in patent applications CN 102051202 and US 2007/080099.

In particular, said hydrotreatment catalyst comprises a support, preferably a mineral support, and at least one metallic element having a hydrodehydrogenating function. Said metallic element having a hydrodehydrogenating function advantageously comprises at least one group VIII element, preferably chosen from the group consisting of nickel and cobalt, and/or at least one group VIB element, preferably chosen from the group consisting of molybdenum and tungsten. The total content of oxides of metallic elements from groups VIB and VIII is preferably between 0.1% and 40% by weight and preferentially from 5% to 35% by weight relative to the total weight of the catalyst. The weight ratio expressed as metal oxide between the group VIB metal(s) relative to the group VIII metal(s) is preferably between 1.0 and 20 and preferably between 2.0 and 10. For example, the hydrotreatment reaction section of step b) of the process comprises a hydrotreatment catalyst comprising between 0.5% and 10% by weight of nickel, preferably between 1% and 8% by weight of nickel, expressed as nickel oxide NiO relative to the total weight of the hydrotreatment catalyst, and between 1.0% and 30% by weight of molybdenum and/or tungsten, preferably between 3.0% and 29% by weight, expressed as molybdenum oxide MoO₃ or tungsten oxide WO₃ relative to the total weight of the hydrotreatment catalyst, on a mineral support.

The support for said hydrotreatment catalyst is advantageously chosen from alumina, silica, silica-aluminas, magnesia, clays and mixtures thereof. Said support may also advantageously contain dopant compounds, notably oxides chosen from boron oxide, in particular boron trioxide, zirconia, ceria, titanium oxide, phosphorus pentoxide and a mixture of these oxides. Preferably, said hydrotreatment catalyst comprises an alumina support, preferably an alumina support doped with phosphorus and optionally boron. When phosphorus pentoxide P₂O₅ is present, its concentration is less than 10% by weight relative to the weight of the alumina and advantageously at least 0.001% by weight relative to the total weight of the alumina. When boron trioxide B₂O₅ is present, its concentration is less than 10% by weight relative to the weight of the alumina and advantageously at least 0.001% relative to the total weight of the alumina. The alumina used may be, for example, a γ(gamma) or η(eta) alumina.

Said hydrotreatment catalyst is, for example, in the form of extrudates.

Advantageously, said hydrotreatment catalyst used in step e) of the process has a specific surface area of greater than or equal to 250 m²/g, preferably greater than or equal to 300 m²/g. The specific surface area of said hydrotreatment catalyst is advantageously less than or equal to 800 m²/g, preferably less than or equal to 600 m²/g, in particular less than or equal to 400 m²/g. The specific surface area of the hydrotreatment catalyst is measured by the BET method, that is to say the specific surface area determined by nitrogen adsorption in accordance with the standard ASTM D 3663-78 established from the Brunauer-Emmett-Teller method described in the periodical The Journal of the American Chemical Society, 60, 309 (1938). Such a specific surface area makes it possible to further improve the removal of the contaminants, in particular of the metals such as silicon.

According to another aspect of the invention, the hydrotreatment catalyst as described above also comprises one or more organic compounds containing oxygen and/or nitrogen and/or sulfur. Such a catalyst is often denoted by the term “additivated catalyst”. Generally, the organic compound is chosen from a compound including one or more chemical functions chosen from carboxylic, alcohol, thiol, thioether, sulfone, sulfoxide, ether, aldehyde, ketone, ester, carbonate, amine, nitrile, imide, oxime, urea and amide functions or else compounds including a furan ring or else sugars.

The preparation of the catalysts for steps a), b) or e) or else e′) is known and generally comprises, when it is a matter of supported catalysts, a step of impregnation of the group VIII metals and of the group VIB metals when present, and optionally of the phosphorus and/or boron on the support, followed by drying, and then optionally calcining. In the case of an additivated catalyst, the preparation generally takes place by simple drying without calcining after introducing the organic compound. The term “calcining” means herein a heat treatment under a gas containing air or oxygen at a temperature of greater than or equal to 200° C. Before their use in a process step, the catalysts are generally subjected to sulfurization so as to form the active species.

In a preferred embodiment of the invention, said hydrotreatment reaction section comprises several fixed-bed reactors, preferentially between two and five, very preferentially between two and four fixed-bed reactors, each containing n catalytic beds, n being an integer greater than or equal to one, preferably between one and ten, preferably between two and five, advantageously operating in series and/or in parallel and/or in permutable (or PRS) mode and/or in “swing” mode. The various optional operating modes, PRS (or lead and lag) mode and swing mode, are well known to those skilled in the art and are advantageously defined hereinabove. The advantage of a hydrotreatment reaction section comprising several reactors lies in optimized treatment of the hydrocarbon cut comprising compounds with a boiling point of less than or equal to 385° C. obtained from step d), while at the same time making it possible to reduce the risks of clogging of the catalytic bed(s) and thus to avoid shutting down the unit due to clogging.

According to a very preferred embodiment of the invention, said hydrotreatment reaction section comprises at least one fixed bed reactor, preferably consists of one reactor or two reactors in series, said fixed-bed reactor(s) containing between one and five catalytic beds arranged in series and each comprising between one and ten hydrotreatment catalysts, of which at least one of said hydrotreatment catalysts advantageously comprises a support and at least one metallic element preferably comprising at least one group VIII element, preferably chosen from nickel and cobalt, and/or at least one group VIB element, preferably chosen from molybdenum and tungsten.

Optionally, step e) may involve a heating section located upstream of the hydrotreatment reaction section and in which the hydrocarbon cut comprising compounds with a boiling point of less than or equal to 385° C. obtained from step d) is heated to reach a temperature suitable for the hydrotreatment, i.e. a temperature of between 250 and 430° C. Said optional heating section may thus comprise one or more exchangers, preferably allowing heat exchange between the hydrocarbon cut comprising compounds with a boiling point of less than or equal to 385° C. obtained from step d) and the hydrotreated effluent, and/or a preheating furnace.

The hydrotreatment step e) advantageously allows optimized treatment of the hydrocarbon cut comprising compounds with a boiling point of less than or equal to 385° C. obtained from step d). It makes it possible notably to eliminate the remaining impurities, notably to eliminate the sulfur compounds and the nitrogen compounds and also the remaining metals.

Hydrocracking Step a′) (Optional)

According to one variant, the process of the invention may comprise a hydrocracking step e′) performed either directly after the hydrotreatment step e), or after step f) of separation on a heavy cut (diesel cut).

The cut comprising compounds with a boiling point of less than or equal to 385° C. comprises a cut comprising compounds with a boiling point below 175° C. (naphtha cut) and a cut comprising compounds with a boiling point above 175° C. and below 385° C. (diesel cut). When it is desired to minimize the yield of the diesel cut and maximize the yield of the naphtha cut, the diesel cut can be at least partly transformed into naphtha cut by hydrocracking, or even part of the heavy naphtha cut into light naphtha, which is the cut generally favoured for a steam cracking unit.

Thus, the process of the invention may comprise a hydrocracking step e′) performed in a hydrocracking reaction section, using at least one fixed bed containing n catalytic beds, n being an integer greater than or equal to 1, each comprising at least one hydrocracking catalyst, said hydrocracking reaction section being fed with said hydrotreated effluent obtained from step e) and/or with the diesel cut comprising compounds with a boiling point above 175° C. and below 385° C. obtained from step f) and a gas stream comprising hydrogen, said hydrocracking reaction section being operated at a temperature of between 250 and 450° C., a partial pressure of hydrogen of between 1.5 and 20.0 MPa abs. and an hourly space velocity of between 0.1 and 10.0 h⁻¹, to obtain a hydrocracked effluent which is sent into the separation step f).

Advantageously, step e′) involves hydrocracking reactions that are well known to those skilled in the art, and more particularly makes it possible to convert the heavy compounds, for example compounds with a boiling point of greater than 175° C., into compounds with a boiling point of less than or equal to 175° C. contained in the hydrotreated effluent obtained from step b). Other reactions, such as the hydrogenation of olefins or of aromatics, hydrodemetallation, hydrodesulfurization, hydrodeazotization, etc. may follow.

Advantageously, said step e′) is performed in a hydrocracking reaction section comprising at least one, preferably between one and five, fixed beds containing n catalytic beds, n being an integer greater than or equal to one, preferably between one and ten, preferably between two and five, said bed(s) each comprising at least one, and preferably not more than ten, hydrocracking catalysts.

The hydrotreatment step e) and the hydrocracking step e′) may advantageously be performed in the same reactor or in different reactors. When they are performed in the same reactor, the reactor comprises several catalytic beds, the first catalytic beds comprising the hydrotreatment catalyst(s) and the following catalytic beds comprising the hydrocracking catalyst(s).

Said hydrocracking reaction section is advantageously operated at a hydrotreatment temperature of between 250 and 450° C., preferably between 320 and 430° C., at a partial pressure of hydrogen of between 1.5 and 20 MPa abs., and at an hourly space velocity (HSV) of between 0.1 and 10.0 h⁻¹, preferably between 0.1 and 5.0 h⁻¹, preferentially between 0.2 and 4 h⁻¹. According to the invention, the “hydrocracking temperature” corresponds to an average temperature in the hydrocracking reaction section of step c). In particular, it corresponds to the weight-average bed temperature (WABT) according to the dedicated terminology, which is well known to those skilled in the art. The hydrocracking temperature is advantageously determined as a function of the catalytic systems, of the equipment and of the configuration thereof that are used. For example, the hydrocracking temperature (or WABT) is calculated in the following manner:

WABT=(Tᵢₙₗₑₜ+2×Tₒᵤₜₗₑₜ)/3

with Tᵢₙₗₑₜ: the temperature of the hydrogenated effluent at the inlet of the hydrocracking reaction section, Tₒᵤₜₗₑₜ: the temperature of the effluent at the outlet of the hydrocracking reaction section.

The hourly space velocity (HSV) is defined here as the ratio of the hourly volume flow rate of the hydrotreated effluent obtained from step e) per volume of catalyst(s). The hydrogen coverage in step e′) is advantageously between 80 and 2000 Nm³ of hydrogen per m³ of feedstock which feeds step e′), and preferably between 200 and 1800 Nm³ of hydrogen per m³ of feedstock which feeds step e′). The hydrogen coverage is defined here as the ratio of the volume flow rate of hydrogen taken under standard temperature and pressure conditions relative to the volume flow rate of feedstock which feeds step e′) (in normal m³, written as Nm³, of H₂ per m³ of feedstock). The hydrogen may consist of a supply and/or of recycled hydrogen obtained in particular from the separation steps c) and d).

Preferably, an additional gas stream comprising hydrogen is advantageously introduced into the inlet of each reactor, in particular operating in series, and/or into the inlet of each catalytic bed from the second catalytic bed of the hydrocracking reaction section. These additional gas streams are also referred to as cooling streams. They make it possible to control the temperature in the hydrocracking reactor in which the reactions involved are generally highly exothermic.

The hydrocracking step e′) can be performed in one or two steps. When it is performed in two steps, the effluent from the first hydrocracking step e′) is separated, making it possible to obtain a cut comprising compounds with a boiling point above 175° C. (cut diesel), which is introduced into the second hydrocracking step. This configuration is particularly suitable when it is desired to produce only a naphtha cut. The operating conditions and catalysts used in the two hydrocracking steps may be identical or different.

These operating conditions used in step e′) of the process according to the invention generally make it possible to obtain conversions per pass, into products having at least 80% by volume of compounds having boiling points of less than or equal to 175° C., preferably below 160° C. and preferably below 150° C., of greater than 15% by weight and even more preferably of between 20% and 80% by weight. When the process is performed in two hydrocracking steps, the conversion per pass in the second step is kept moderate so as to maximize the selectivity towards compounds of the naphtha cut (with a boiling point of less than or equal to 175° C., in particular between 80 and less than or equal to 175° C.). The conversion per pass is limited by the use of a high recycle ratio over the second hydrocracking step loop. This ratio is defined as the ratio of the feed flow rate of step f) to the flow rate of the feedstock of step a); preferentially, this ratio is between 0.2 and 4, preferably between 0.5 and 2.5.

The hydrocracking step e′) thus does not necessarily make it possible to transform all the compounds with a boiling point of greater than 175° C. (diesel cut) into compounds with a boiling point of less than or equal to 175° C. (naphtha cut). After the fractionation step f), there may therefore remain a greater or lesser proportion of compounds with a boiling point above 175° C. To increase the conversion, at least part of this unconverted cut can be recycled as described below into step e′). Another part can be purged. Depending on the operating conditions of the process, said purge may be between 0 and 10% by weight of the cut comprising compounds with a boiling point above 175° C. relative to the ingoing feedstock, and preferably between 0.5% and 5% by weight.

In accordance with the invention, the hydrocracking step e′) is performed in the presence of at least one hydrocracking catalyst.

The hydrocracking catalyst(s) used in the hydrocracking step e′) are conventional hydrocracking catalysts known to those skilled in the art, of bifunctional type combining an acid function with a hydro-dehydrogenating function and optionally at least one binder matrix. The acid function is provided by supports having large surface areas (generally 150 to 800 m²/g) having surface acidity, such as halogenated (notably chlorinated or fluorinated) aluminas, combinations of boron and aluminium oxides, amorphous silica-aluminas and zeolites. The hydro-dehydrogenating function is provided by at least one metal from group VIB of the Periodic Table and/or at least one metal from group VIII.

Preferably, the hydrocracking catalyst(s) used in step e′) comprise a hydro-dehydrogenating function comprising at least one metal from group VIII chosen from iron, cobalt, nickel, ruthenium, rhodium, palladium and platinum, and preferably from cobalt and nickel. Preferably, said catalyst(s) also comprise at least one metal from group VIB chosen from chromium, molybdenum and tungsten, alone or as a mixture, and preferably from molybdenum and tungsten. Hydro-dehydrogenating functions of NiMo, NiMoW or NiW type are preferred.

Preferably, the content of metal from group VIII in the hydrocracking catalyst(s) is advantageously between 0.5% and 15% by weight and preferably between 1% and 10% by weight, the percentages being expressed as weight percentage of oxides relative to the total weight of the catalyst.

Preferably, the content of metal from group VIB in the hydrocracking catalyst(s) is advantageously between 5% and 35% by weight and preferably between 10% and 30% by weight, the percentages being expressed as weight percentage of oxides relative to the total weight of the catalyst.

The hydrocracking catalyst(s) used in step e′) may also optionally comprise at least one promoter element deposited on the catalyst and chosen from the group formed by phosphorus, boron and silicon, optionally at least one element from group VIIA (chlorine and fluorine preferred), optionally at least one element from group VIIB (manganese preferred), and optionally at least one element from group VB (niobium preferred).

Preferably, the hydrocracking catalyst(s) used in step e′) comprise at least one amorphous or poorly crystallized porous mineral matrix of oxide type chosen from aluminas, silicas, silica-aluminas, aluminates, alumina-boron oxide, magnesia, silica-magnesia, zirconia, titanium oxide or clay, alone or as a mixture, and preferably aluminas or silica-aluminas, alone or as a mixture.

Preferably, the silica-alumina contains more than 50% by weight of alumina, preferably more than 60% by weight of alumina.

Preferably, the hydrocracking catalyst(s) used in step e′) also optionally comprise a zeolite chosen from Y zeolites, preferably from USY zeolites, alone or in combination with other zeolites from among beta, ZSM-12, IZM-2, ZSM-22, ZSM-23, SAPO-11, ZSM-48 or ZBM-30 zeolites, alone or as a mixture. Preferably, the zeolite is USY zeolite alone.

When said catalyst comprises a zeolite, the content of zeolite in the hydrocracking catalyst(s) is advantageously between 0.1% and 80% by weight, preferably between 3% and 70% by weight, the percentages being expressed as a percentage of zeolite relative to the total weight of the catalyst.

A preferred catalyst comprises, and preferably consists of, at least one metal from group VIB and optionally at least one non-noble metal from group VIII, at least one promoter element, and preferably phosphorus, at least one Y zeolite and at least one alumina binder.

An even more preferred catalyst comprises, and preferably consists of, nickel, molybdenum, phosphorus, a USY zeolite, and optionally also a beta zeolite, and alumina.

Another preferred catalyst comprises, and preferably consists of, nickel, tungsten, alumina and silica-alumina.

Another preferred catalyst comprises, and preferably consists of, nickel, tungsten, a USY zeolite, alumina and silica-alumina.

Said hydrocracking catalyst is, for example, in the form of extrudates.

According to another aspect of the invention, the hydrocracking catalyst as described above also comprises one or more organic compounds containing oxygen and/or nitrogen and/or sulfur. Such a catalyst is often denoted by the term “additivated catalyst”. Generally, the organic compound is chosen from a compound including one or more chemical functions chosen from carboxylic, alcohol, thiol, thioether, sulfone, sulfoxide, ether, aldehyde, ketone, ester, carbonate, amine, nitrile, imide, oxime, urea and amide functions or else compounds including a furan ring or else sugars.

Separation Step f)

According to the invention, the treatment process comprises a separation step f), fed with the hydrotreated effluent obtained from step e) or with the hydrocracked effluent obtained from the optional step e′) to obtain at least one gaseous effluent and a hydrotreated liquid hydrocarbon effluent.

The separation section of step f) is advantageously operated in separation equipment that is well known (separating vessels which may be operated at various pressures and temperatures, pumps, heat exchangers, stripping columns, distillation columns, etc.).

The gaseous effluent obtained on conclusion of step f) advantageously comprises hydrogen, preferably comprises at least 90% by volume, preferably at least 95% by volume, of hydrogen. Advantageously, said gaseous effluent may be at least partly recycled into the selective hydrogenation step a) and/or the hydroconversion step b) and/or the hydrotreatment step e) and/or the optional hydrocracking step e′), the recycling system possibly comprising a purification section.

The gaseous effluent obtained on conclusion of step f) may also comprise light hydrocarbons, notably ethane, propane and butane, which may advantageously be sent separately or as a mixture to one or more furnaces of the steam cracking step h).

Step f) makes it possible in particular to remove the gases dissolved in the liquid hydrocarbon effluent, for instance ammonia, hydrogen sulfide and light hydrocarbons containing 1 to 4 carbon atoms.

The fractionation step f) is advantageously performed at a pressure of less than or equal to 1.0 MPa abs., preferably between 0.1 and 1.0 MPa abs.

According to one embodiment, step f) may be performed in a section advantageously comprising at least one stripping column equipped with a reflux circuit comprising a reflux vessel. Said stripping column is fed with the hydrotreated effluent obtained from step e) and with a steam stream. The hydrotreated effluent obtained from step e) may optionally be heated before entering the stripping column. Thus, the lightest compounds are entrained to the top of the column and into the reflux circuit comprising a reflux vessel in which a gas/liquid separation is performed. The gaseous phase which comprises the light hydrocarbons is withdrawn from the reflux vessel as a gas stream. The hydrotreated liquid hydrocarbon effluent which is the cut comprising compounds with a boiling point of less than or equal to 385° C. hydrotreated (naphtha cut and diesel cut) is advantageously withdrawn at the bottom of the stripping column.

According to other embodiments, the separation step f) may involve a stripping column followed by a distillation column or only a distillation column.

In a particular embodiment, the separation step f) may include a fractionation, making it possible to obtain, besides a gas stream, a naphtha cut comprising compounds with a boiling point of less than or equal to 175° C., preferably between 80 and 175° C., and a diesel cut comprising compounds with a boiling point above 175° C. and below 385° C.

The naphtha cut can be sent, totally or partly, to a steam cracking unit and/or to the naphtha pool obtained from conventional petroleum feedstocks; it can also be sent into the recycling step g).

The diesel cut may also be sent, totally or partly, either to a steam cracking unit, or to a jet fuel and diesel pool obtained from conventional petroleum feedstocks, or sent into the recycling step g), or be introduced into the hydrocracking step e′) when it is present.

In another particular embodiment, the naphtha cut comprising compounds with a boiling point of less than or equal to 175° C. is fractionated to give a heavy naphtha cut comprising compounds with a boiling point of between 80 and 175° C. and a light naphtha cut comprising compounds with a boiling point below 80° C. At least a portion of said heavy naphtha cut may be sent to an aromatic complex including at least one step of reforming of the naphtha for the purpose of producing aromatic compounds. The heavy naphtha cut may also be sent at least partly into the steam cracking step h) described below. According to this embodiment, at least a portion of the light naphtha cut is sent into the steam cracking step h) described below.

Step g) (Optional) of Recycling the Hydrotreated Liquid Hydrocarbon Effluent Obtained from Step f)

The process according to the invention may comprise the recycling step g), in which a fraction of the hydrotreated liquid hydrocarbon effluent obtained from the separation step f) is recovered to constitute a recycle stream which is sent upstream of or directly into at least one of the reaction steps of the process according to the invention, in particular into the optional selective hydrogenation step a) and/or the hydroconversion step b) and/or the hydrotreatment step e). Optionally, a fraction of the recycle stream may be sent into the optional pretreatment step a0).

Preferably, at least a fraction of the hydrotreated liquid hydrocarbon effluent obtained from the separation step f) feeds the hydrotreatment step e).

Advantageously, the amount of the recycle stream is adjusted so that the weight ratio between the recycle stream and the feedstock comprising a pyrolysis oil, i.e. the feedstock to be treated feeding the overall process, is less than or equal to 10, preferably less than or equal to 5, and preferentially greater than or equal to 0.001, preferably greater than or equal to 0.01, and preferably greater than or equal to 0.1. Very preferably, the amount of the recycle stream is adjusted so that the weight ratio between the recycle stream and the feedstock comprising a pyrolysis oil is between 0.2 and 5.

Depending on the feedstocks treated, the recycling of a portion of the product obtained into or upstream of at least one of the reaction steps of the process according to the invention advantageously makes it possible, firstly, to dilute the impurities and, secondly, to control the temperature in the reaction step(s) in which the reactions involved may be highly exothermic.

According to a preferred embodiment of the invention, the process for treating a feedstock comprising a pyrolysis oil comprises, and preferably consists of, the sequence of steps as follows, and preferably in the order given:
b) hydroconversion,
c) separation,
d) fractionation,
e) hydrotreatment,
f) separation,
to produce an effluent, at least part of which is suitable for treatment in a steam cracking unit.

According to a second preferred embodiment of the invention, the process for treating a feedstock comprising a pyrolysis oil comprises, and preferably consists of, the sequence of steps as follows, and preferably in the order given:
b) hydroconversion,
c) separation,
d) fractionation with recycling of at least part of the hydrocarbon cut comprising compounds with a boiling point above 385° C. into step b),
e) hydrotreatment,
f) separation,
to produce an effluent, at least part of which is suitable for treatment in a steam cracking unit.

According to a third preferred embodiment of the invention, the process for treating a feedstock comprising a pyrolysis oil comprises, and preferably consists of, the sequence of the steps as follows, and preferably in the order given:
b) hydroconversion,
c) separation,
d) fractionation with recycling of at least part of the hydrocarbon cut comprising compounds with a boiling point above 385° C. into step b),
e) hydrotreatment,
e′) hydrocracking,
f) separation,
to produce an effluent, at least part of which is suitable for treatment in a steam cracking unit.

According to a fourth preferred embodiment of the invention, the process for treating a feedstock comprising a plastics and/or SRF pyrolysis oil comprises, and preferably consists of, the sequence of steps as follows, and preferably in the order given:
a) selective hydrogenation,
b) hydroconversion,
c) separation,
d) fractionation with recycling of at least part of the hydrocarbon cut comprising compounds with a boiling point above 385° C. into step b),
e) hydrotreatment, e′) hydrocracking,
f) separation,
g) recycling of at least part of the hydrotreated liquid hydrocarbon effluent obtained from step
f) into steps a) and/or b) and/or e),
to produce an effluent, at least part of which is suitable for treatment in a steam cracking unit.

Said hydrotreated liquid hydrocarbon effluent or a fraction of said effluent thus obtained by treatment according to the process of the invention of a pyrolysis oil has a composition that is compatible with the specifications for a feedstock entering a steam cracking unit. In particular, the composition of the hydrocarbon effluent or of said hydrocarbon stream(s) is preferably such that:
the total content of metallic elements is less than or equal to 5.0 ppm by weight, preferably less than or equal to 2.0 ppm by weight, preferentially less than or equal to 1.0 ppm by weight and preferably less than or equal to 0.5 ppm by weight, with:
a content of iron (Fe) element of less than or equal to 100 ppb by weight,
a content of silicon (Si) element of less than or equal to 1.0 ppm by weight, preferably less than or equal to 0.6 ppm by weight, and
the sulfur content is less than or equal to 500 ppm by weight, preferably less than or equal to 200 ppm by weight,
the nitrogen content is less than or equal to 100 ppm by weight, preferably less than or equal to 50 ppm by weight and preferably less than or equal to 5 ppm by weight,
the total content of chlorine element is less than or equal to 10 ppm by weight, preferably less than 1.0 ppm by weight,
the content of asphaltenes is less than or equal to 5.0 ppm by weight,
the content of olefinic compounds (monoolefins and diolefins) is less than or equal to 5.0% by weight, preferably less than or equal to 2.0% by weight and preferably less than or equal to 0.1% by weight.

The contents are given as relative weight concentrations, weight percentages (%), parts per million (ppm) by weight or parts per billion (ppb) by weight, relative to the total weight of the stream under consideration.

The process according to the invention thus makes it possible to treat the SRF and/or plastics pyrolysis oils to obtain an effluent which can be totally or partly injected into a steam cracking unit.

Steam Cracking Step h) (Optional)

The hydrotreated liquid hydrocarbon effluent obtained from step f) can be sent, totally or partly, into a steam cracking step h).

Advantageously, the gas fraction(s) obtained from the separation step c) and/or f) and containing ethane, propane and butane, may also be totally or partly sent into the steam cracking step h).

Said steam cracking step h) is advantageously performed in at least one pyrolysis furnace at a temperature of between 700 and 900° C., preferably between 750 and 850° C., and under a pressure of between 0.05 and 0.3 MPa relative. The residence time of the hydrocarbon compounds is generally less than or equal to 1.0 second (noted as s), preferably between 0.1 and 0.5 s. Steam is advantageously introduced upstream of the optional steam cracking step h) and after the separation (or fractionation). The amount of water introduced, advantageously in the form of steam, is advantageously between 0.3 and 3.0 kg of water per kg of hydrocarbon compounds entering step h). The optional step h) is preferably performed in a plurality of pyrolysis furnaces in parallel, so as to adapt the operating conditions to the various streams feeding step h) and notably obtained from steps c) and/or f), and also to manage the tube decoking times. A furnace comprises one or more tubes arranged in parallel. A furnace can also denote a group of furnaces operating in parallel. For example, a furnace may be dedicated to the cracking of the naphtha cut comprising compounds with a boiling point of less than or equal to 175° C.

The effluents from the various steam cracking furnaces are generally recombined before separation for the purpose of constituting an effluent. It is understood that the steam cracking step h) includes the steam cracking furnaces but also the substeps associated with steam cracking that are well known to those skilled in the art. These substeps may notably include heat exchangers, columns and catalytic reactors and recycling into the furnaces. A column generally makes it possible to fractionate the effluent for the purpose of recovering at least one light fraction comprising hydrogen and compounds containing 2 to 5 carbon atoms, and a fraction comprising pyrolysis gasoline, and optionally a fraction comprising pyrolysis oil. Columns make it possible to separate the various constituents of the fractionation light fraction so as to recover at least one cut rich in ethylene (C2 cut) and a cut rich in propylene (C3 cut) and optionally a cut rich in butenes (C4 cut). The catalytic reactors notably make it possible to perform selective hydrogenations of the C2, C3 or even C4 cuts and of the pyrolysis gasoline. The saturated compounds, notably the saturated compounds containing 2 to 4 carbon atoms, are advantageously recycled into the steam cracking furnaces so as to increase the overall yields of olefins.

This steam cracking step h) makes it possible to obtain at least one effluent containing olefins comprising 2, 3 and/or 4 carbon atoms (i.e. C2, C3 and/or C4 olefins), in satisfactory contents, in particular greater than or equal to 30% by weight, notably greater than or equal to 40% by weight, or even greater than or equal to 50% by weight of total olefins comprising 2, 3 and 4 carbon atoms relative to the weight of the steam cracking effluent under consideration. Said C2, C3 and C4 olefins may then be advantageously used as polyolefin monomers.

According to one or more preferred embodiments of the invention, taken separately or combined, the process for treating a feedstock comprising an SRF and/or plastics pyrolysis oil comprises, and preferably consists of, the sequence of steps described above, preferably in the order given:
b) hydroconversion,
c) separation,
d) fractionation,
e) hydrotreatment,
f) separation,
h) steam cracking.

According to a preferred mode, the process for treating a feedstock comprising an SRF and/or plastics pyrolysis oil comprises, and preferably consists of, the sequence of the steps described above, and preferably in the order given:
a) selective hydrogenation,
b) hydroconversion,
c) separation,
d) fractionation with recycling of at least part of the hydrocarbon cut comprising compounds with a boiling point above 385° C. into step b),
e) hydrotreatment,
e′) hydrocracking,
f) separation,
h) steam cracking of at least the other part of the hydrotreated liquid hydrocarbon effluent obtained from step f).

Analysis Methods Used

The analysis methods and/or standards used for determining the characteristics of the various streams, in particular of the feedstock to be treated and of the effluents, are known to those skilled in the art. They are in particular listed below for information purposes. Other reputed equivalent methods may also be used, notably equivalent IP, EN or ISO methods:

TABLE 1

| Description | Methods |
|---|---|
| Density at 15° C. | ASTM D4052 |
| Sulfur content | ISO 20846 |
| Nitrogen content | ASTM D4629 |
| Acid number | ASTM D664 |
| Bromine number | ASTM D1159 |
| Diolefin content based on the maleic anhydride | MAV method (1) |
| value | |
| Content of oxygen-based compounds | Combustion + Infrared |
| Content of paraffins | UOP990-11 |
| Content of naphthenes and olefins | UOP990-11 |
| Content of aromatics | UOP990-11 |
| Content of halogens | ASTM D7359 |
| Chloride content | ASTM D7536 |
| Content of metals: | ASTM D5185 |
| P | |
| Fe | |
| Si | |
| Na | |
| B | |
| Simulated distillation | ASTM D2887 |

(1) MAV method described in the article: C. López-García et al., Near Infrared Monitoring of Low Conjugated Diolefins Content in Hydrotreated FCC Gasoline Streams, Oil & Gas Science and Technology - Rev. IFP, Vol. 62 (2007), No. 1, pages 57-68

### LIST OF FIGURES

The information regarding the elements referenced in FIG. 1 enables a better understanding of the invention, without said invention being limited to the particular embodiments illustrated in FIG. 1. The various embodiments presented may be used alone or in combination with each other, without any limit to the combinations.

FIG. 1 represents the scheme of a particular embodiment of the process of the present invention, comprising:

an optional step a) of selective hydrogenation of a hydrocarbon feedstock obtained from pyrolysis, in the presence of a hydrogen-rich gas 2 and optionally of an amine supplied by the stream 3, performed in at least one fixed-bed reactor including at least one selective hydrogenation catalyst, to obtain an effluent 4;
a step b) of hydroconversion of the effluent 4 obtained from step a), in the presence of hydrogen 5, performed in at least one ebullated-bed, entrained-bed and/or moving-bed reactor including at least one hydroconversion catalyst, to obtain a hydroconverted effluent 6;
a step c) of separation of the effluent 6 performed in the presence of an aqueous washing solution 7, making it possible to obtain at least one fraction 8 comprising hydrogen, an aqueous fraction 9 containing dissolved salts, and a hydrocarbon liquid fraction 10;
a step d) of fractionation of the liquid hydrocarbon fraction 10 making it possible to obtain at least one gaseous fraction 11, a hydrocarbon cut 12 comprising compounds with a boiling point of less than or equal to 385° C. and a hydrocarbon cut 13 comprising compounds with a boiling point above 385° C. which is preferably at least partly recycled into step b);
a step e) of hydrotreatment of at least part of the hydrocarbon cut 12 comprising compounds with a boiling point of less than or equal to 385° C. obtained from step d), in the presence of hydrogen 14, performed in at least one fixed-bed reactor including at least one hydrotreatment catalyst, to obtain a hydrotreated effluent 15;
a step f) of separation of the effluent 15 making it possible to obtain at least one fraction 16 comprising hydrogen and a hydrotreated liquid hydrocarbon effluent 17.

At the end of step f), at least a part of the hydrotreated liquid hydrocarbon 17 is sent to a steam cracking process (not represented).

Optionally, part of said hydrotreated liquid hydrocarbon effluent 17 constitutes a recycle stream 17a, 17b and 17c which feeds steps a) and/or b) and/or e), respectively.

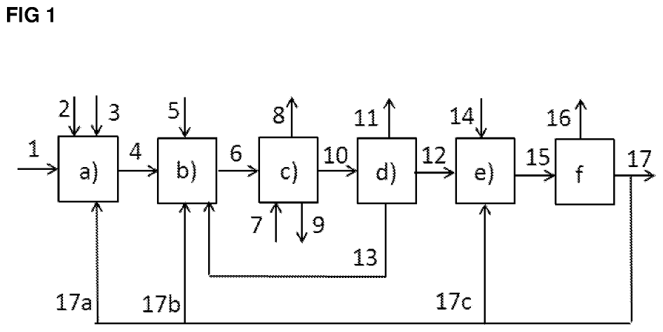

Only the main steps, with the main streams, are shown in FIG. 1, so as to allow a better understanding of the invention. It is clearly understood that all the items of equipment required for the functioning are present (vessels, pumps, exchangers, furnaces, columns, etc.), even if they are not shown. It is also understood that hydrogen-rich gas streams (supply or recycle), as described above, may be injected into the inlet of each reactor or catalytic bed or between two reactors or two catalytic beds. Means well known to those skilled in the art for purifying and recycling hydrogen may also be used.

### EXAMPLES

### Example 1 (in Accordance with the Invention)

Feedstock 1 treated in the process is an SRF pyrolysis oil having the characteristics indicated in Table 2.

TABLE 2
Characteristics of the feedstock

| | | | Pyrolysis |
|---|---|---|---|
| Description | Methods | Unit | oil |
| Density at 15° C. | ASTM D4052 | g/cm³ | 0.91 |
| Sulfur content | ISO 20846 | ppm by | 3500 |
| | | weight | |
| Nitrogen content | ASTM D4629 | ppm by | 2900 |
| | | weight | |
| Acid number | ASTM D664 | mg KOH/g | 15 |
| Bromine number | ASTM D1159 | g/100 g | 60 |
| Diolefin content based on the | MAV method | weight % | 7.0 |
| maleic anhydride value | | | |
| Content of oxygen-based | Combustion + | weight % | 1.0 |
| compounds | Infrared | | |
| Content of paraffins | UOP990-11 | weight % | 15 |
| Content of naphthenes and | UOP990-11 | weight % | 25 |
| olefins | | | |
| Content of aromatics | UOP990-11 | weight % | 60 |
| Content of halogens | ASTM D7359 | ppm by | 400 |
| | | weight | |
| Chloride content | ASTM D7536 | ppm by | 300 |
| | | weight | |
| Content of metals: | ASTM D5185 | | |
| P | | ppm by | 20 |
| | | weight | |
| Fe | | ppm by | 30 |
| | | weight | |
| Si | | ppm by | 500 |
| | | weight | |
| Na | | ppm by | 2 |
| | | weight | |
| B | | ppm by | 2 |
| | | weight | |
| Simulated distillation | ASTM D2887 | | |
| 0% | | ° C. | 50 |
| 10% | | ° C. | 120 |
| 30% | | ° C. | 145 |
| 50% | | ° C. | 170 |
| 70% | | ° C. | 230 |
| 90% | | ° C. | 320 |
| 100% | | ° C. | 405 |

The feedstock 1 is subjected directly (without a selective hydrogenation step a)) to a hydroconversion step b) performed in an ebullated bed and in the presence of hydrogen 5 and of a hydrotreatment catalyst of NiMo type on alumina, under the conditions indicated in Table 3.

TABLE 3
conditions of the hydroconversion step b)

| | | |
|---|---|---|
| Hydroconversion temperature | ° C. | 400 |
| Partial pressure of hydrogen | MPa abs | 9.0 |
| H₂/HC (volume coverage of hydrogen | Nm³/m³ | 300 |
| relative to the feedstock volume) | | |
| HSV (flow rate by volume of | h⁻¹ | 1.0 |
| feedstock/volume of catalysts) | | |

The effluent 6 obtained from the hydroconversion step b) is sent to step the separation c) and then to the fractionation step d). Table 4 gives the yields of the various fractions obtained at the end of the fractionation step d) relative to the feedstock 1 entering the process chain.

TABLE 4
yields for the various products and fractions
obtained at the end of step b)

| | | |
|---|---|---|
| H₂S + NH₃ | weight % | 0.35 |
| C1-C4 | weight % | 3.70 |
| PI-175° C. Fraction | weight % | 59.0 |
| 175° C.-385° C. Fraction | weight % | 35.0 |
| 385° C.+ Fraction | weight % | 3.0 |
| Total | weight % | 101.05 |

The liquid cut comprising compounds with a boiling point of less than or equal to 385° C. (naphtha cut and diesel cut) is then sent to step e) of hydrotreatmnent and in the presence of hydrogen and of a hydrotreatmnent catalyst of the NiMo type on alumina under the conditions shown in Table 5.

TABLE 5
conditions of the hydrotreatment step b)

| | | |
|---|---|---|
| Hydrotreatment temperature | ° C. | 350 |
| Partial pressure of hydrogen | MPa abs | 9.0 |
| H₂/HC (volume coverage of hydrogen | Nm³/m³ | 300 |
| relative to the feedstock volume) | | |
| HSV (flow rate by volume of | h⁻¹ | 0.5 |
| feedstock/volume of catalysts) | | |

The effluent 17 obtained from the hydrotreatment step e) is subjected to a step f) of separation and fractionation.

Table 6 gives the overall yields relative to the feedstock 1 entering the process chain for the various fractions obtained at the end of the separation and fractionation step f) (which comprises a stripping column and a distillation column).

TABLE 6
yields for the various products and fractions
obtained at the end of step f)

| | | |
|---|---|---|
| H₂S + NH₃ | weight % | 0.50 |
| C1-C4 | weight % | 4.50 |
| PI-175° C. Fraction | weight % | 60.5 |
| 175° C.-385° C. Fraction | weight % | 32.5 |
| 385° C.+ Fraction | weight % | 2.5 |
| Total | weight % | 100.50 |

The compounds H₂S and NH₃ are mainly eliminated in the form of salts in the aqueous phase removed in the separation step d).

The characteristics of the PI-175° C. and 175° C.-385° C. liquid fractions obtained after the separation and fractionation step f) are shown in Table 7:

TABLE 7
characteristics of the PI-175° C. and 175° C.-385° C. fractions

| | | PI-175° C. | 175° C.-385° C. |
|---|---|---|---|
| | Unit | Fraction | Fraction |
| Density at 15° C. | g/cm³ | 0.762 | 0.813 |
| (ASTM D4052) | | | |
| Content of: | | | |
| Sulfur (ASTM D5453) | ppm by | <2 | <2 |
| | weight | | |
| Nitrogen (ASTM D4629) | ppm by | <5 | <10 |
| | weight | | |
| Fe (ASTM D5185) | ppb by | Not | 25 |
| | weight | detected | |
| Total metals | ppm by | Not | 1 |
| (ASTM D5185) | weight | detected | |
| Chlorine (ASTM D7536) | ppb by | Not | <25 |
| | weight | detected | |
| Paraffins (UOP990-11) | weight % | 45 | 30 |
| Naphthenes (UOP990-11) | weight % | 50 | 60 |
| Olefins (UOP990-11) | weight % | 5 | 10 |
| Aromatics (UOP990-11) | weight % | 1 | 2 |
| Simulated distillation | | | |
| (ASTM D2887) in % | | | |
| 0 | ° C. | 25 | 175 |
| 5 | ° C. | 38 | 185 |
| 10 | ° C. | 59 | 210 |
| 30 | ° C. | 90 | 240 |
| 50 | ° C. | 118 | 275 |
| 70 | ° C. | 140 | 310 |
| 90 | ° C. | 163 | 360 |
| 95 | ° C. | 175 | 385 |
| 100 | ° C. | 180 | 395 |

The PH-175° C. and 175° C.-385° C. liquid fractions both have compositions that are compatible with a steam cracking unit, since:
they have very low contents of chlorine element (respectively, an undetected content and a content of 25 ppb by weight), which are below the limit required for a steam cracking feedstock;
the contents of metals, in particular of iron (Fe), are also very low (contents of metals not detected for the PI-175° C. fraction and <1 ppm by weight for the 175° C.-385° C. fraction;
contents of Fe not detected for the PH-175° C. fraction and of 25 ppb by weight for the 175° C.-385° C. fraction), which are below the limits required for a steam cracking feedstock (s 5.0 ppm by weight, very preferably s 1 ppm by weight for metals; s 100 ppb by weight for Fe);
finally, they contain sulfur (<2 ppm by weight for the PI-175° C. fraction and <2 ppm by weight for the 175° C.-385° C. fraction) and nitrogen (<5 ppm by weight for the PI-175° C. fraction and <10 ppm by weight for the 175° C.-385° C. fraction) with contents that are very much lower than the limits required for a steam cracking feedstock (≤500 ppm by weight, preferably 200 ppm by weight for S and N).

The PI-175° C. and 175° C.-385° C. liquid fractions obtained are thus subsequently sent into a steam cracking step h) (cf. Table 8).

TABLE 8
conditions of the steam cracking step

| | | PI-175° C. | 175° C.-385° C. + |
|---|---|---|---|
| | Unit | Fraction | Fraction |
| Furnace outlet pressure | MPa abs | 0.2 | 0.2 |
| Furnace outlet temperature | ° C. | 835 | 820 |
| Steam/feedstock ratio | kg/kg | 0.6 | 0.8 |
| Furnace residence time | s | 0.25 | 0.2 |

The effluents from the various steam cracking furnaces are subjected to a separation step which enables recycling of the saturated compounds into the steam cracking furnaces and the production of the yields presented in Table 9 (yield=mass % of product relative to the mass of the PI-175° C. and 175° C.-385° C. fraction upstream of the steam cracking step, noted as weight %).

TABLE 9
yields for the steam cracking step

| | | PI-175° C. | 175° C.-385° C. |
|---|---|---|---|
| Fraction | | Fraction | Fraction |
| H₂, CO, C1 | weight % | 19.0 | 12.0 |
| Ethylene | weight % | 33.0 | 26.0 |
| Propylene | weight % | 16.0 | 16.0 |
| C4 cut | weight % | 9.0 | 9.0 |
| Pyrolysis gasoline | weight % | 17.0 | 19.0 |
| Pyrolysis oil | weight % | 6.0 | 18.0 |

Considering the yields obtained for the PI-175° C. and 175° C.-385° C. liquid fractions during the pyrolysis oil treatment process at the outlet of the hydroconversion and hydrotreatment steps (cf. Table 6), it is possible to determine the overall yields relative to the feedstock 1 entering the process chain for the products obtained from the steam cracking step h) relative to the initial feedstock of SRF pyrolysis oil type introduced into step a):

TABLE 10
yields of products obtained from the step of
steam cracking of the PI-175° C. fraction
and of the 175° C.-385° C. fraction

| | | |
|---|---|---|
| H₂S + NH₃ | weight % | 0.50 |
| H2, CO, C1 | weight % | 16.0 |
| C2 | weight % | 0.6 |
| C3 | weight % | 1.7 |
| C4 | weight % | 1.8 |
| Ethylene | weight % | 28.7 |
| Propylene | weight % | 15.1 |
| C4 cut | weight % | 8.4 |
| Pyrolysis gasoline | weight % | 16.7 |
| Pyrolysis oil | weight % | 9.5 |
| 385° C.+ Fraction | weight % | 2.5 |
| Total | weight % | 101.50 |

When the PI-175° C. and 175-385° C. fractions are sent to the steam cracking unit, the process according to the invention makes it possible to achieve overall mass yields of ethylene and propylene, respectively, of 28.7% and 15.1% relative to the mass amount of initial feedstock of pyrolysis oil type.

Furthermore, the specific sequence of steps upstream of the steam cracking step makes it possible to limit the formation of coke and to avoid the corrosion problems which would have appeared had the chlorine not been removed.

### Example 2 (in Accordance with the Invention)

The feedstock 1 treated in the process is a plastics pyrolysis oil having the characteristics indicated in Table 11.

TABLE 11
feedstock characteristics

| | | | Pyrolysis |
|---|---|---|---|
| Description | Methods | Unit | oil |
| Density at 15° C. | ASTM D4052 | g/cm³ | 0.82 |
| Sulfur content | ISO 20846 | ppm by | 2500 |
| | | weight | |
| Nitrogen content | ASTM D4629 | ppm by | 730 |
| | | weight | |
| Acid number | ASTM D664 | mg KOH/g | 1.5 |
| Bromine number | ASTM D1159 | g/100 g | 80 |
| Diolefin content based on | MAV method | weight % | 10.0 |
| the maleic anhydride value | | | |
| Content of oxygen-based | Combustion + | weight % | 1.0 |
| compounds | Infrared | | |
| Content of paraffins | UOP990-11 | weight % | 45 |
| Content of naphthenes and | UOP990-11 | weight % | 45 |
| olefins | | | |
| Content of aromatics | UOP990-11 | weight % | 10 |
| Content of halogens | ASTM D7359 | ppm by | 350 |
| | | weight | |
| Chloride content | ASTM D7536 | ppm by | 320 |
| | | weight | |
| Content of metals: | ASTM D5185 | | |
| P | | ppm by | 10 |
| | | weight | |
| Fe | | ppm by | 25 |
| | | weight | |
| Si | | ppm by | 45 |
| | | weight | |
| Na | | ppm by | 2 |
| | | weight | |
| B | | ppm by | 2 |
| | | weight | |
| Simulated distillation | ASTM D2887 | | |
| 0% | | ° C. | 40 |
| 10% | | ° C. | 98 |
| 30% | | ° C. | 161 |
| 50% | | ° C. | 232 |
| 70% | | ° C. | 309 |
| 90% | | ° C. | 394 |
| 100% | | ° C. | 432 |

The feedstock 1 is subjected to a selective hydrogenation step a) performed in a fixed-bed reactor and in the presence of hydrogen 2 and of a selective hydrogenation catalyst of NiMo type on alumina, under the conditions indicated in Table 12.

TABLE 12
conditions of the selective hydrogenation step a)

| | | |
|---|---|---|
| Temperature | ° C. | 180 |
| Partial pressure of hydrogen | MPa abs | 9.0 |
| H₂/HC (volume coverage of hydrogen | Nm³/m³ | 50 |
| relative to the feedstock volume) | | |
| HSV (flow rate by volume of | h⁻¹ | 0.5 |
| feedstock/volume of catalysts) | | |

On conclusion of the selective hydrogenation step a), the diolefin content in the feedstock has been significantly reduced.

The effluent 4 obtained from the selective hydrogenation step a) is subjected directly, without separation, to a hydroconversion step b) performed in an ebullated bed and in the presence of hydrogen 5 and of a hydrotreatment catalyst of NiMo type on alumina under the conditions presented in Table 13.

TABLE 13
conditions of the hydroconversion step b)

| | | |
|---|---|---|
| Hydroconversion temperature | ° C. | 380 |
| Partial pressure of hydrogen | MPa abs | 9.0 |
| H₂/HC (volume coverage of hydrogen | Nm³/m³ | 300 |
| relative to the feedstock volume) | | |
| HSV (volume flow rate of feedstock | h⁻¹ | 1.5 |
| in step b)/volume of catalysts) | | |

The effluent 6 obtained from the hydroconversion step b) is sent to the separation step c) and then to the fractionation step d). Table 14 gives the yields of the various fractions obtained at the end of the fractionation step d) relative to the feedstock 1 entering the process chain.

TABLE 14
yields for the various products and fractions obtained
at the outlet of the fractionation step d)

| | | |
|---|---|---|
| H₂S + NH₃ | weight % | 0.4 |
| C1-C4 | weight % | 1.0 |
| PI-150° C. Fraction | weight % | 28.4 |
| 150° C.+ Fraction | weight % | 70.9 |
| Total | weight % | 100.7 |

The liquid cut comprising compounds with a boiling point of less than or equal to 385° C. (naphtha cut PI-150° C. and diesel cut 150° C.+) is then sent into the hydrotreatment step e) and in the presence of hydrogen and a hydrotreatmnent catalyst of the NiMo on alumina type under the conditions shown in Table 15.

TABLE 15
conditions of the hydrotreatment step e)

| | | |
|---|---|---|
| Hydrotreatment temperature | ° C. | 350 |
| Partial pressure of hydrogen | MPa abs | 9.0 |
| H₂/HC (volume coverage of hydrogen | Nm³/m³ | 300 |
| relative to the feedstock volume) | | |
| HSV (flow rate by volume of | h⁻¹ | 1 |
| feedstock/volume of catalysts) | | |

The effluent 17 obtained on conclusion of the hydrotreatmnent step e) is subjected to a step f) of separation and fractionation.

Table 16 gives the overall yields, relative to the feedstock 1 entering the process chain, for the various fractions obtained at the end of the separation and fractionation step f) (which comprises a stripping column and a distillation column).

TABLE 16
yields for the various products and fractions obtained at
the outlet of the separation and fractionation step f)

| | | |
|---|---|---|
| H₂S + NH₃ | weight % | 0.5 |
| C1-C4 | weight % | 1.1 |
| PI-150° C. Fraction | weight % | 28.6 |
| 150° C.+ Fraction | weight % | 70.7 |
| Total | weight % | 100.9 |

The compounds 1H₂S and NH₃ are mainly eliminated in the form of salts in the aqueous phase removed in the separation step d).

The characteristics of the PI-150° C. and 150° C.+ liquid fractions obtained after the separation and fractionation step f) are shown in Table 17:

TABLE 17
characteristics of the PI-150° C and 150° C.+ fractions
after step f) of separation and fractionation

| | | PI-150° C. | 150° C.+ |
|---|---|---|---|
| | | Fraction | Fraction |
| Density at 15° C. (ASTM D4052) | g/cm³ | 0.750 | 0.820 |
| Content of: | | | |
| Sulfur (ASTM D5453) | ppm by | <2 | <2 |
| | weight | | |
| Nitrogen (ASTM D4629) | ppm by | <5 | <10 |
| | weight | | |
| Fe (ASTM D5185) | ppb by | Not | <50 |
| | weight | detected | |
| Total metals (ASTM D5185) | ppm by | Not | <1 |
| | weight | detected | |
| Chlorine (ASTM D7536) | ppb by | Not | <25 |
| | weight | detected | |
| Paraffins (UOP990-11) | weight % | 75 | 70 |
| Naphthenes (UOP990-11) | weight % | 25 | 28 |
| Olefins (UOP990-11) | weight % | Not | Not |
| | | detected | detected |
| Aromatics (UOP990-11) | weight % | <1 | 2 |
| Simulated distillation | | | |
| (ASTM D2887) in % | | | |
| 0 | ° C. | 25 | 140 |
| 5 | ° C. | 32 | 162 |
| 10 | ° C. | 40 | 174 |
| 30 | ° C. | 82 | 226 |
| 50 | ° C. | 108 | 281 |
| 70 | ° C. | 126 | 346 |
| 90 | ° C. | 142 | 395 |
| 95 | ° C. | 146 | 404 |
| 100 | ° C. | 160 | 425 |

The PI-150° C. and 150° C.+ liquid fractions both have compositions that are compatible with a steam cracking unit, since:
they do not contain any olefins (monoolefins and diolefins);
they have very low contents of chlorine element (respectively, an undetected content and a content of 25 ppb by weight), which are below the limit required for a steam cracking feedstock;
the contents of metals, in particular of iron (Fe), are also very low (contents of metals not detected for the PI-150° C. fraction and <1 ppm by weight for the 150° C.+ fraction; contents of Fe not detected for the PI-150° C. fraction and <50 ppb by weight for the 150° C.+ fraction), which are below the limits required for a steam cracking feedstock (s 5.0 ppm by weight, very preferably ≤1 ppm by weight for metals; s 100 ppb by weight for Fe);
finally, they contain sulfur (<2 ppm by weight for the PI-150° C. fraction and <2 ppm by weight for the 150° C.+ fraction) and nitrogen (<5 ppm by weight for the PI-150° C. fraction and <10 ppm by weight for the 150° C.+ fraction) with contents that are very much lower than the limits required for a steam cracking feedstock (s 500 ppm by weight, preferably s 200 ppm by weight for S and N).

The PI-150° C. and 150° C.+ liquid fractions obtained are then advantageously sent into a steam cracking step h).

1. Process for treating a feedstock comprising a solid recovery fuel and/or plastics pyrolysis oil, comprising:
a) optionally, a selective hydrogenation step performed in a reaction section fed at least with said feedstock and a gas stream comprising hydrogen, in the presence of at least one selective hydrogenation catalyst, at a temperature of between 100 and 280° C., a partial pressure of hydrogen of between 1.0 and 20.0 MPa abs. and an hourly space velocity of between 0.3 and 10.0 h⁻¹, to obtain a hydrogenated effluent;
b) a hydroconversion step performed in a hydroconversion reaction section, using at least one ebullated-bed reactor, entrained-bed reactor or moving-bed reactor, comprising at least one hydroconversion catalyst, said hydroconversion reaction section being fed at least with said feedstock or with said hydrogenated effluent obtained on conclusion of step a) and a gas stream comprising hydrogen, said hydroconversion reaction section being operated at a temperature of between 250 and 450° C., a partial pressure of hydrogen of between 1.0 and 20.0 MPa abs. and an hourly space velocity of between 0.05 and 10.0 h⁻¹, to obtain a hydroconverted effluent;
c) a separation step, fed with the hydroconverted effluent obtained from step b) and an aqueous solution, said step being performed at a temperature of between 50 and 450° C., to obtain at least one gaseous effluent, an aqueous effluent and a hydrocarbon effluent;
d) a step of fractionating all or some of the hydrocarbon effluent obtained from step c), to obtain at least one gas stream, a hydrocarbon cut comprising compounds with a boiling point of less than or equal to 385° C. and a hydrocarbon cut comprising compounds with a boiling point above 385° C.,
e) a hydrotreatment step performed in a hydrotreatment reaction section, using at least one fixed-bed reactor containing n catalytic beds, n being an integer greater than or equal to 1, each comprising at least one hydrotreatment catalyst, said hydrotreatment reaction section being fed with at least some of said hydrocarbon cut comprising compounds with a boiling point of less than or equal to 385° C. obtained from step d) and a gas stream comprising hydrogen, said hydrotreatment reaction section being operated at a temperature of between 250 and 430° C., a partial pressure of hydrogen of between 1.0 and 20.0 MPa abs. and an hourly space velocity of between 0.1 and 10.0 h⁻¹, to obtain a hydrotreated effluent;
f) a separation step, fed with the hydrotreated effluent obtained from step e) to obtain at least a gaseous effluent and a hydrotreated liquid hydrocarbon effluent.

2. Process according to claim 1, comprising said selective hydrogenation step a).

3. Process according to claim 1, in which the hydrocarbon cut comprising compounds with a boiling point above 385° C. obtained from step d) is at least partly recycled into step b).

4. Process according to claim 1, comprising a step a0) of pretreating the feedstock, said pretreatment step being performed upstream of the optional selective hydrogenation step a) or upstream of the hydroconversion step b) and comprises a filtration step and/or a step of washing with water and/or an adsorption step.

5. Process according to claim 1, in which the hydrotreated liquid hydrocarbon effluent obtained from step f) is sent into a steam cracking step h) performed in at least one pyrolysis furnace at a temperature of between 700 and 900° C. and at a pressure of between 0.05 and 0.3 MPa relative.

6. Process according to claim 1, which also comprises a recycling step g) in which a fraction of the hydrotreated liquid hydrocarbon effluent obtained from the separation step f) is sent into the optional selective hydrogenation step a) and/or the hydroconversion step b) and/or the hydrotreatment step e).

7. Process according to claim 1, in which the separation step f) comprises a fractionation making it possible to obtain, in addition to a gas stream, a naphtha cut comprising compounds with a boiling point of less than or equal to 175° C., and a diesel cut comprising compounds with a boiling point above 175° C. and below 385° C.

8. Process according to claim 1, which also comprises a hydrocracking step e′) performed in a hydrocracking reaction section, using at least one fixed bed containing n catalytic beds, n being an integer greater than or equal to 1, each comprising at least one hydrocracking catalyst, said hydrocracking reaction section being fed at least with said hydrotreated effluent obtained from step e) and/or with the diesel cut comprising compounds with a boiling point above 175° C. and below 385° C. obtained from step f) and a gas stream comprising hydrogen, said hydrocracking reaction section being operated at a temperature of between 250 and 450° C., a partial pressure of hydrogen of between 1.5 and 20.0 MPa abs. and an hourly space velocity of between 0.1 and 10.0 h⁻¹, to obtain a hydrocracked effluent which is sent into the separation step f).

9. Process according to claim 1, in which the separation step f) also comprises fractionation of the naphtha cut comprising compounds with a boiling point of less than or equal to 175° C. into a light naphtha cut comprising compounds with a boiling point below 80° C. and a heavy naphtha cut comprising compounds with a boiling point of between 80 and 175° C.

10. Process according to claim 9, in which at least part of said heavy naphtha cut is sent to an aromatic complex including at least one naphtha reforming step and/or in which at least part of the light naphtha cut is sent into the steam cracking step h).

11. Process according to claim 1, in which said selective hydrogenation catalyst of step a) comprises a support chosen from alumina, silica, silica-aluminas, magnesia, clays and mixtures thereof and a hydro-dehydrogenating function comprising either at least one group VIII element and at least one group VIB element, or at least one group VIII element.

12. Process according to claim 1, in which, when step b) is performed in an ebullated bed or in a moving bed, said hydroconversion catalyst of step b) comprises a supported catalyst comprising a group VIII metal chosen from the group formed by Ni, Pd, Pt, Co, Rh and/or Ru, optionally a group VIB metal chosen from the group Mo and/or W, on an amorphous mineral support chosen from the group formed by alumina, silica, silica-aluminas, magnesia, clays and mixtures of at least two of these minerals, and when step b) is performed in an entrained bed, said hydroconversion catalyst of step b) comprises a dispersed catalyst containing at least one element chosen from the group formed by Mo, Fe, Ni, W, Co, V and Ru.

13. Process according to claim 1, in which said hydrotreatment catalyst of step e) comprises a support chosen from the group consisting of alumina, silica, silica-aluminas, magnesia, clays and mixtures thereof and a hydro-dehydrogenating function comprising at least one group VIII element and/or at least one group VIB element.

14. Process according to claim 8, in which said hydrocracking catalyst of step e′) comprises a support chosen from halogenated aluminas, combinations of boron and aluminium oxides, amorphous silica-aluminas and zeolites and a hydro-dehydrogenating function comprising at least one group VIB metal chosen from chromium, molybdenum and tungsten, alone or as a mixture, and/or at least one group VIII metal chosen from iron, cobalt, nickel, ruthenium, rhodium, palladium and platinum.

15. Process according to claim 1, in which the feedstock has the following properties:
a content of aromatic compounds of between 0 and 90% by weight,
a content of halogenated compounds of between 2 and 5000 ppm by weight,
a content of metallic elements of between 10 and 10 000 ppm by weight,
including a content of iron element of between 0 and 100 ppm by weight,
a content of silicon element of between 0 and 1000 ppm by weight.

16. Product which may be obtained via the process according to claim 1.

17. Product according to claim 16, which includes, relative to the total weight of the product:
a total content of metal elements of less than or equal to 5.0 ppm by weight,
including a content of iron element of less than or equal to 100 ppb by weight,
a content of silicon element of less than or equal to 1.0 ppm by weight,
a sulfur content of less than or equal to 500 ppm by weight,
a nitrogen content of less than or equal to 100 ppm by weight,
a content of chlorine element of less than or equal to 10 ppm by weight.
